SEQUENCE LISTING (105 sequences)

SEQ ID NO: 1 - protein, 150 aa, Corynebacterium glutamicum
MYCPFCQHDHSKVIDSRVIDAGSAIRRRRECSKCEGRFTTIEKAVLLVVKRNGVTEPFSREKVVTGVRRACQGRDVSDDALKRLAQQVEETVRSNGSSQVRANDIGLAILDPLRELDEVAYLRFASVYKSFDSADDFEKEIRLMRRRGRD

SEQ ID NO: 2 - protein, 253 aa, Corynebacterium glutamicum
MAIEKKPAGARGSRGSRTVKTLPNGKPDPASLSDRQRRILEVIRDAVVLRGYPPSIREIGDAAGLQSTSSVAYQLKELEKKGFLRRDPNKPRAVDVRHLPETESRSSKAATQAKSKAPQAGVHDPELAGQTSFVPVVGKIAAGSPITAEQNIEEYYPLPAEIVGDGDLFMLQVVGESMRDAGILTGDWVVVRSQPVAEQGEFVAAMIDGEATVKEFHKDSSGIWLLPHNDTFAPIPAENAEIMGKVVSVMRKL

SEQ ID NO: 3 - protein, 379 aa, Corynebacterium glutamicum
MAQNKGSDKSQTEKRKGGLGRGLAALIPSGPSNSPGLGGGAADIILGGTVGARTAAAPKRESTPAAPAPEAPAQAAPQHTEATKPEVVPEPAAPAPTQSAQQEAPQAQPAQQSEFGASYLEIPIEQIRPNPQQPRHEFDPQALDELVHSISEFGLMQPIVVRRSEDGYELIMGERRWRASKRAGLEVIPAIVRETEDSAMLRDALLENIHRVQLNPLEEAAAYQQLLEEFGVTQAELADKLGRSRPVITNMIRLLGLPVNVQTKVAAGVLSAGHARALLGLKAGEDAQDTLATRIIAEGLSVRATEELVLLHNRGDQDEEKKPREKAATPEVFTRAAESLADNLDTKVSVMMGKKKGKLVVEFGDKDDFERIMSLIQGQ

SEQ ID NO: 4 - protein, 164 aa, Corynebacterium glutamicum
MTSENSESQDIWLTDEQQDVWLDVWTMRIGLPARLDAQLKEAAGVSHFEYFTMAQISMAPEHRVRMSELAELSDMTLSHLSRVVTRLEKAGWVKRVPDPDDGRATVAVLTDSGWEKVKATAPGHVKEVRRLVFDDLTPEELKVMGTAMKKIVNRLDMSNRLPRV

SEQ ID NO: 5 - protein, 212 aa, Corynebacterium glutamicum
MEAAGTEILMPRRRPAQQRSRERFNRILTAARSVLVDLGFESFTFDEVAKRAEVPIGTLYQFFANKYVLICELDRVDTAEAVAELKKFSDQVPALQWPDILDEFIEHLARLWRDDPSRRAVWHAIQSTPATRATAAATEKEMLEIIAEVMRPLARGAGYEERMSLAGLLVHTVSSLLNYAVRDVNSSEEDFDSIVEEIKRMLISYLFSVATG

SEQ ID NO: 6 - protein, 202 aa, Corynebacterium glutamicum
MTLEAIEDNATRLILERGFDNVTIEDICAEAGISKRTFFNYVESKESVAIGHTAKLPTDEEREAFLATRHENIIDTVFDLVINLFGNHDNSKSGVAGDIMRRRKEIRVKHPELAVQHFARFHQAREGLEHLIVEYFEKWPGSQHLDEPADREAIAIVGLLISVMLQGSREWHDMPQGTQADFQACCRKAIKNTFLLRGGFSE

SEQ ID NO: 7 - protein, 192 aa, Corynebacterium glutamicum
MHFEELNNESCGSRGSGEFRGRPGRRHAQRHAEGHGHGHHHGRRPGRGRGGRAGRGDLRNVILVLLEAESMHGYQIITTISEQTEGNWTPSPGTIYPTLSMLEDEGLISISHEMGRKMARLTEEGAQEVAKNKDAWGSILEAYRNPESREVRVFNIRSEFHKVREAAKAAPDDKAEQIIEILRRAADDIKRL

SEQ ID NO: 8 - protein, 116 aa, Corynebacterium glutamicum
MTSVIPEQRNNPFYRDSATIASSDHTERGEWVTQAKCRNGDPDALFVRGAAQRRAAAICRHCPVAMQCCADALDNKVEFGVWGGLTERQRRALLRKKPHITNWAEYLAQGGEIAGV

SEQ ID NO: 9 - protein, 246 aa, Corynebacterium glutamicum
MPSETMKPAVASTLAATSTGRRPGRPTQRILSVESIVERTLNIAGREGFAAVTMNRLARDMGVTPRALYNHVLNRQEIIDRVWVRIIDDIKVPDLDPDNWRQSIHTLWSSLRDQFRETPRVLLVALDEQISTQGTSPLRIAGAEESLKFLTDIGLSLKEATIIREMMMADVFSFTLTSDYTFDNRPEGEKPDVFAPVPKPWLDENPDVEAPLTRKAVEESVSTSDELFGYMVEARIAYIEKLLAAK

SEQ ID NO: 10 - protein, 405 aa, Corynebacterium glutamicum
MAVKTLKDLLDEGVDGRHVIVRSDFNVPLNDDREITDKGRIIASLPTLKALSEGGAKVIVMAHLGRPKGEVNEKYSLAPVAEALSDELGQYVALAADVVGEDAHERANGLTEGDILLLENVRFDPRETSKDEAERTAFAQELAALAADNGAFVSDGFGVVHRAQTSVYDIAKLLPHYAGGLVETEISVLEKIAESPEAPYVVVLGGSKVSDKIGVIEALAAKADKIIVGGGMCYTFLAAQGHNVQQSLLQEEMKATCTDLLARFGDKIVLPVDLVAASEFNKDAEKQIVDLDSIPEGWMSLDIGPESVKNFGEVLSTAKTIFWNGPMGVFEFAAFSEGTRGIAQAIIDATAGNDAFSVVGGGDSAASVRVLGLNEDGFSHISTGGGASLEYLEGKELPGVAILAQ

SEQ ID NO: 11 - protein, 335 aa, Corynebacterium glutamicum
MNLKNPETPDRNLAMELVRVTEAAALASGRWVGRGMKNEGDGAAVDAMRQLINSVTMKGVVVIGEGEKDEAPMLYNGEEVGTGFGPEVDIAVDPVDGTTLMAEGRPNAISILAAAERGTMYDPSSVFYMKKIAVGPEAAGKIDIEAPVAHNINAVAKSKGINPSDVTVVVLDRPRHIELIADIRRAGAKVRLISDGDVAGAVAAAQDSNSVDIMMGTGGTPEGIITACAMKCMGGEIQGILAPMNDFERQKAHDAGLVLDQVLHTNDLVSSDNCYFVATGVTNGDMLRGVSYRANGATTRSLVMRAKSGTIRHIESVHQLSKLQEYSVVDYTTAT

SEQ ID NO: 12 - protein, 344 aa, Corynebacterium glutamicum
MPIATPEVYNEMLDRAKEGGFAFPAINCTSSETINAALKGFAEAESDGIIQFSTGGAEFGSGLAVKNKVKGAVALAAFAHEAAKSYGINVALHTDHCQKEVLDEYVRPLLAISQERVDRGELPLFQSHMWDGSAVPIDENLEIAQELLAKAKAANIILEVEIGVVGGEEDGVEAKAGANLYTSPEDFEKTIDAIGTGEKGRYLLAATFGNVHGVYKPGNVKLRPEVLLEGQQVARKKLGLADDALPFDFVFHGGSGSEKEKIEEALTYGVIKMNVDTDTQYAFTRPIVSHMFENYNGVLKIDGEVGNKKAYDPRSYMKKAEQSMSERIIESCQDLKSVGKTTSK

SEQ ID NO: 13 - protein, 328 aa, Corynebacterium glutamicum
MNSPQNVSTKKVTVTGAAGQISYSLLWRIANGEVFGTDTPVELKLLEIPQALGGAEGVAMELLDSAFPLLRNITITADANEAFDGANAAFLVGAKPRGKGEERADLLANNGKIFGPQGKAINDNAADDIRVLVVGNPANTNALIASAAAPDVPASRFNAMMRLDHNRAISQLATKLGRGSAEFNNIVVWGNHSATQFPDITYATVGGEKVTDLVDHDWYVEEFIPRVANRGAEIIEVRGKSSAASAASSAIDHMRDWVQGTEAWSSAAIPSTGAYGIPEGIFVGLPTVSRNGEWEIVEGLEISDFQRARIDANAQELQAEREAVRDLL

SEQ ID NO: 14 - protein, 259 aa, Corynebacterium glutamicum
MARKPLIAGNWKMNLDHQQAIGTVQKLAFALPKEYFEKVDVAVTVPFTDIRSVQTLVEGDKLEVTFGAQDVSQHESGAYTGEVSASMLAKLNCSWVVVGHSERREYHNESDELVAAKAKAALSNGISPIVCVGEPLEIREAGTHVEYVVEQTRKSLAGLDAAELANTVIAYEPVWAIGTGKVASAADAQEVCKAIRGLIVELAGDEVAEGLRILYGGSVKAETVAEIVGQPDVDGGLVGGASLDGEAFAKLAANAASVA

SEQ ID NO: 15 - DNA, 453 bp, Corynebacterium glutamicum
gtgtactgcccattttgccaacatgatcattcaaaagtcattgactcccgcgtcattgacgccggaagcgccattcgcaggcgccgcgagtgcagcaaatgcgaaggccgtttcaccaccatcgaaaaagctgttctcctcgttgttaaaagaaacggcgtcactgaaccgttcagtcgagaaaaagtagtcaccggtgtccgtcgtgcatgccaaggccgcgacgtatcagatgacgcgttgaaacgcctagctcagcaagtggaagaaacagtccgcagcaacggaagctctcaagtacgcgctaacgatattggtttagccattctcgatccactgagagaactcgacgaggtagcgtacctacgctttgcctctgtgtataagtcttttgacagtgctgacgactttgaaaaagaaatccgcctcatgcgcagacgcggaagggactag

SEQ ID NO: 16 - DNA, 762 bp, Corynebacterium glutamicum
atggcaatcgaaaagaagccagcaggtgcacgaggcagtcgcggtagccgcacagttaagaccttgcccaacggaaaaccagatccagcaagtttgtcagataggcagcgcaggattttagaggttatccgagatgctgtggttttgaggggttatccaccaagcattagggaaattggtgatgctgcaggacttcaatccacttcttccgttgcttaccagcttaaagagctagagaagaagggcttcctgcgcagggaccctaataagcctcgcgcggtggatgttcgccaccttccagaaactgaaagccgttcctccaaggctgctacacaggcaaagagcaaggcccctcaggccggggtccatgatcctgagttagctggccagacctcatttgtcccagtggtgggcaaaattgccgctggtagcccgatcaccgctgagcagaacatcgaagagtactacccactccccgcagaaatcgtcggagacggtgacttgttcatgctccaggttgttggcgagtccatgagggatgctggcatcctcaccggcgactgggttgttgttcgttcccagccggtcgctgagcagggcgagttcgtcgcggcaatgattgacggtgaagccaccgtgaaggaattccacaaggattcatctggcatctggctcctgccacacaacgatacgtttgccccaattcctgctgagaatgcagaaatcatgggcaaggttgtttccgtgatgcgcaagctttaa

SEQ ID NO: 17 - DNA, 1140 bp, Corynebacterium glutamicum
atggctcagaacaagggttccgacaagtcccagacggaaaagcgtaagggcggtctggggcgcggtctggccgcacttattccctcaggaccaagtaattccccaggtcttggtggcggtgcggctgacatcattttgggcggtaccgtgggtgcgcgtactgctgcagctcccaagcgtgagtccacaccagcagctcctgcacctgaggctcctgcgcaggccgctccacaacacactgaggccacaaagccagaggtagttccagagccagcagctcctgctccaacgcagtcagcacagcaggaagcgccgcaggcacagccagcacagcagtctgagttcggcgcatcctaccttgagatcccgatcgagcagatccgccccaacccgcagcagcctcgccatgagtttgatccgcaggcacttgacgagttggtgcattcgatcagcgagttcggcctcatgcagccgatcgtggtgcgcaggtccgaggatggctatgagctcatcatgggtgagcgtcgttggcgtgcatccaagcgagctggccttgaggttatcccggcgattgtccgtgaaactgaagacagcgcgatgctgcgcgacgcccttttggaaaatatccacagggtgcagctgaatcctttggaagaggcagccgcctaccagcagttgctggaggagttcggtgtcacccaggcagagctggccgataagctgggccgttcccgtccggtaatcaccaatatgattcgtctgctgggccttccagtcaacgtgcagaccaaggtggcagccggtgtgctgtctgcaggccatgcacgcgcattgctggggctcaaggccggcgaggatgctcaggacaccctggcgacccgaatcatcgctgagggcctgtctgtgcgtgctactgaggaattggtgctgctgcacaaccgtggtgatcaggatgaggagaagaagccacgcgaaaaggctgcaactcctgaggtctttacccgtgcggctgagtccttggcggataatttggataccaaggtctcggtgatgatgggtaagaagaagggcaagctcgtggtggaattcggcgacaaggatgatttcgagcgcatcatgtccttgatccagggccaataa

SEQ ID NO: 18 - DNA, 495 bp, Corynebacterium glutamicum
atgacaagtgagaattccgaatcccaggacatttggctaaccgatgagcaacaagatgtgtggctcgatgtgtggacaatgcgaatcggcctgcctgctcgcttggatgctcaactgaaagaagctgcgggtgtcagccactttgagtacttcaccatggcgcagatttctatggccccggaacatcgggtgcgcatgagtgagcttgctgagctgtccgatatgacgctatcgcatctatctagagtggttactcgcctagaaaaggctggctgggtgaagcgtgttcccgatcctgatgatggtcgcgccaccgttgctgtgctcacggactctgggtgggagaaagttaaagcaacagcccctggtcatgtgaaggaagtgcgtcgtttggtgtttgacgatctcactccagaagaactcaaggtaatgggcaccgcaatgaagaagattgtgaaccgactcgatatgtccaacaggctgccccgggtgtag

SEQ ID NO: 19 - DNA, 639 bp, Corynebacterium glutamicum
ttggaagcggcaggcactgaaattttaatgcctcgccgccgtccggcacagcagcgcagtcgtgaacgattcaatcgaatcctcaccgctgcgcgttcagtgcttgtcgatctaggttttgaatcgttcacgtttgatgaagtcgctaagcgtgcagaggtaccgatcggcacgctgtaccaattctttgccaataagtatgtattgatctgcgaattggatcgtgtggataccgcagaagctgtcgcggagttgaagaaattctccgatcaggttcctgcgttgcagtggccggatatccttgatgaattcattgagcacttggctaggctctggcgcgatgatccgtctcggcgggccgtgtggcatgccatccagtccacgccggcaactcgtgcgacagctgcggcgacggaaaaagagatgctggaaatcatcgcggaagttatgcgcccgcttgcccgcggtgccggctacgaggagcgcatgtcactggcgggattgctggtgcacacggtaagttccctgcttaactatgccgtgcgtgatgtcaatagttccgaagaggatttcgacagcatcgtggaagaaatcaaacgaatgctgatttcttacctcttctccgtggctactggatag

SEQ ID NO: 20 - DNA, 609 bp, Corynebacterium glutamicum
atgaccctggaagcgatagaagataacgcaaccaggctcattctggagcgtggcttcgacaatgtcacaatcgaagacatctgcgcagaggcagggatatccaagcgcacattctttaactacgtggagtccaaagagtctgtggccatcgggcacacagccaagctcccaacggatgaagaacgtgaagcattcctggctacgcgtcatgaaaatattatcgatactgtatttgacctggtaatcaacctctttggcaaccacgacaactccaagtctggagttgcaggcgacattatgcgtcgacgcaaagagatccgggtgaagcatccagaactggcagtgcaacatttcgccaggttccaccaagcacgcgaagggctagaacacctaattgttgagtacttcgaaaaatggccaggctcccaacatctagatgagcctgcagatcgagaagcaatcgccatagttggcctgctgatctcggtcatgcttcaaggttctcgtgaatggcacgacatgccacaaggcacgcaagctgatttccaagcctgctgtcgcaaagcaattaaaaatacttttcttcttagaggtggattttcagaatga

SEQ ID NO: 21 - DNA, 579 bp, Corynebacterium glutamicum
atgcattttgaagaactaaacaatgaatcttgtggatcacgcggatccggagaattcaggggaaggccgggcaggcgtcatgctcaacggcacgctgaagggcacggacatggacatcatcacggcaggcgacccggacgtggtcgcggtggacgtgctggcagaggcgatctgcgcaatgtgattttggtgctgctggaagctgagtcaatgcacggctaccagatcatcaccaccatcagtgagcaaacagaaggtaactggactccaagcccaggaaccatctatccaaccttgtccatgcttgaagatgaaggcctgatttccatctcccatgaaatgggcagaaaaatggcgcgccttacagaagaaggcgcgcaggaagtggcaaagaacaaggatgcgtggggatcaattctggaggcttatcgcaatccagaatcccgagaggtgcgggtgtttaacattcgctctgagtttcacaaggtcagggaagcagcgaaagctgctcccgacgataaagcagagcaaataatcgagattttaaggagagcagcagatgacatcaagagactataa

SEQ ID NO: 22 - DNA, 351 bp, Corynebacterium glutamicum
atgacgtctgtgattccagagcagcgcaacaaccccttttatagggacagcgccacaattgcttcctcggaccacacagagcgtggtgagtgggtcactcaggcaaagtgtcgaaatggcgacccagatgcattgtttgttcgtggtgcagcgcaacgccgagcagcagcaatttgccgccactgccctgtagccatgcagtgctgcgccgatgccttagataacaaggtggaattcggagtctggggaggcctgaccgagcgccagcgccgtgcattgcttcgaaagaagccgcacattactaactgggctgaatatttggctcaggggggcgagatcgccggggtttaa

SEQ ID NO: 23 - DNA, 741 bp, Corynebacterium glutamicum
atgcctagcgaaactatgaaaccagccgtagcgtcaactctggcggccacttccacgggacgtcgtcctggacgccccacccaacgtatcctttccgtcgaatccatagtggagcgcactttaaacattgccggccgcgaaggattcgctgccgtgaccatgaaccgcctcgcccgagacatgggtgtcacccctcgcgcactgtataaccatgtgctaaatcgtcaagaaatcattgatcgcgtctgggtgcgcatcatcgatgatatcaaggtgcccgatcttgatccggacaattggcggcaatctattcatacgctgtggagctcattgcgcgaccaattccgtgagactccacgtgttcttctggtcgcgctggatgaacagatctctactcagggcacttccccactgcgaatcgcgggtgcggaggagtccttgaagttcttgactgatatcgggctgtccctcaaggaagcaaccatcatccgggagatgatgatggctgatgtcttcagcttcaccctgacttctgactacacctttgacaatcgtccagagggcgaaaagccggatgtgtttgctccggttcctaagccatggcttgatgagaacccagatgtggaagcgccactgacccgtaaagcagtcgaagagtccgtctcaacttctgacgaactcttcggctacatggtggaggctcgcattgcttatattgaaaagctgcttgccgccaaatag

SEQ ID NO: 24 - DNA, 1218 bp, Corynebacterium glutamicum
atggctgttaagaccctcaaggacttgctcgacgaaggcgtagacggacgccacgtcatcgttcgatctgacttcaatgttcccctcaacgatgaccgcgagatcaccgataagggccgaatcattgcctccctaccaacccttaaagcactgagcgaaggtggcgcaaaggtcatcgtcatggctcaccttggccgcccaaagggcgaggtcaacgagaagtactccctcgcacctgtcgctgaggcactctccgatgagcttggccagtacgttgcacttgccgcagacgttgttggcgaagacgcacacgagcgcgcaaacggcctgaccgagggcgacatcctgctcctggagaacgtgcgcttcgacccacgcgaaacctccaaggacgaggcagagcgcaccgctttcgctcaggagctcgcagctcttgcagcagacaacggcgcattcgtttctgacggcttcggtgttgtccaccgcgcacagacctccgtctacgacattgcaaagttgctgccacactacgctggcggactggtagagaccgagatttccgttctggaaaagatcgcagaatcaccagaggcaccatacgtagtggttctcggtggatccaaggtctctgacaagatcggtgttattgaggcgctggctgccaaggctgacaagatcatcgtcggtggcggcatgtgctacaccttcctcgcagctcagggacacaacgttcagcagtccctcctgcaggaagaaatgaaggctacctgcaccgacctgctcgcacgcttcggtgacaagatcgttctcccagttgacctggttgcagcatccgaatttaacaaggacgcagagaagcagatcgttgacctggactccatcccagaaggctggatgtctcttgacatcggaccagagtccgtcaagaacttcggtgaggttctcagcaccgctaagaccatcttctggaacggcccaatgggcgtgttcgagttcgcagcattctctgaaggcacccgcggcatcgcccaggccatcatcgatgcaactgcaggcaacgacgcattctccgttgttggcggtggcgactccgcagcatccgttcgcgtgctcggcctgaacgaagacggcttctcccacatctccaccggtggtggcgcatccctcgagtaccttgaaggcaaggaactcccaggcgttgcaattctcgctcagtaa

SEQ ID NO: 25 - DNA, 1008 bp, Corynebacterium glutamicum
atgaacctaaagaaccccgaaacgccagaccgtaaccttgctatggagctggtgcgagttacggaagcagctgcactggcttctggacgttgggttggacgtggcatgaagaatgaaggcgacggtgccgctgttgacgccatgcgccagctcatcaactcagtgaccatgaagggcgtcgttgttatcggcgagggcgaaaaagacgaagctccaatgctgtacaacggcgaagaggtcggaaccggctttggacctgaggttgatatcgcagttgacccagttgacggcaccaccctgatggctgagggtcgccccaacgcaatttccattctcgcagctgcagagcgtggcaccatgtacgatccatcctccgtcttctacatgaagaagatcgccgtgggacctgaggccgcaggcaagatcgacatcgaagctccagttgcccacaacatcaacgcggtggcaaagtccaagggaatcaacccttccgacgtcaccgttgtcgtgcttgaccgtcctcgccacatcgaactgatcgcagacattcgtcgtgcaggcgcaaaggttcgtctcatctccgacggcgacgttgcaggtgcagttgcagcagctcaggattccaactccgtggacatcatgatgggcaccggcggaaccccagaaggcatcatcactgcgtgcgccatgaagtgcatgggtggcgaaatccagggcatcctggccccaatgaacgatttcgagcgccagaaggcacacgacgctggtctggttcttgatcaggttctgcacaccaacgatctggtgagctccgacaactgctacttcgtggcaaccggtgtgaccaacggtgacatgctccgtggcgtttcctaccgcgcaaacggcgcaaccacccgttccctggttatgcgcgcaaagtcaggcaccatccgccacatcgagtctgtccaccagctgtccaagctgcaggaatactccgtggttgactacaccaccgcgacctaa

SEQ ID NO: 26 - DNA, 1035 bp, Corynebacterium glutamicum
atgcctatcgcaactcccgaggtctataacgagatgctcgatcgtgctaaggaaggcggattcgccttcccagccatcaactgcacctcctcggaaaccatcaacgcagctctcaagggcttcgcagaggctgaatctgacggaatcatccagttctccaccggtggtgcagagttcggttccggcctggcagtaaagaacaaggtcaagggcgcagttgcgcttgcagccttcgcccacgaggcagcaaagagctacggcatcaacgttgctctgcacactgaccactgccagaaggaagtcctggacgagtacgtccgcccactgctggctatctcccaggagcgcgtcgaccgcggcgagcttccactgttccagtcccacatgtgggatggttccgctgtcccaatcgacgagaacctcgaaatcgcacaggagctgctggctaaggccaaggcagcgaacatcatcttggaagttgagatcggtgttgtcggtggcgaagaagacggcgttgaggctaaggctggcgcaaacctctacacctccccagaagactttgagaagaccatcgatgcaatcggcaccggtgagaagggccgctacctgctagcagctaccttcggtaacgtccacggcgtttacaagccaggcaacgtcaagctgcgcccagaggtcctccttgagggccagcaggttgcacgcaagaagcttggacttgcagacgacgcacttccattcgacttcgtcttccacggtggctcaggctccgagaaggaaaagatcgaagaggcgctgacctacggcgtcatcaagatgaacgttgatactgacacccagtacgcattcacccgcccaatcgtctcccacatgtttgagaactacaacggcgttctcaagatcgacggcgaggtcggaaacaagaaggcttacgacccacgctcttacatgaagaaggctgagcagagcatgtctgagcgcattatcgagtcttgccaggacctcaagtctgttggaaagaccacctctaagtaa

SEQ ID NO: 27 - DNA, 987 bp, Corynebacterium glutamicum
atgaattccccgcagaacgtctccaccaagaaggtcaccgtcaccggcgcagctggtcaaatctcttattcactgttgtggcgcatcgccaacggtgaagtattcggcaccgacacccctgtagaactgaaacttctggagatccctcaggctcttggcggggcagagggtgtggctatggaacttctggattctgccttccccctcctgcgaaacatcaccatcaccgcggatgccaatgaggcattcgacggcgctaatgcggcgtttttggtcggtgcgaagcctcgcggaaaaggcgaagagcgcgcagatttgctggctaacaacggcaagattttcggacctcaaggtaaagctatcaatgacaacgccgcagatgacattcgtgtcctagttgttggaaacccagcgaacaccaacgcgttgattgcttcagctgcggccccagatgttccagcatcccgcttcaacgcaatgatgcgccttgatcacaaccgtgcgatctcccagctggccaccaagcttggccgtggatctgcggaatttaacaacattgtggtctggggaaatcactccgcaacccagttcccagacatcacctacgcaaccgttggtggagaaaaggtcactgacctggttgatcacgattggtatgtggaggagttcattcctcgcgtggctaaccgtggcgctgaaatcattgaggtccgtggaaagtcttctgcagcttctgcagcatcctctgcgattgatcacatgcgcgattgggtacagggcaccgaggcgtggtcctctgcggcaattccttccaccggtgcatacggcattcctgagggcatttttgtcggtctgccaaccgtatcccgcaacggtgagtgggaaatcgttgaaggcctggagatttccgatttccagcgcgcccgcatcgacgcgaatgctcaggaattgcaggccgagcgcgaggcagtgcgcgacttgctctaa

SEQ ID NO: 28 - DNA, 780 bp, Corynebacterium glutamicum
atggcacgtaagccacttatcgctggtaactggaagatgaacctggatcaccagcaggcaatcggcactgttcagaagcttgcattcgcccttccaaaggaatacttcgagaaggttgacgttgcagtcaccgttcctttcactgacatccgctccgtccagactctcgttgagggcgacaagcttgaggtcactttcggtgctcaggacgtctcccagcacgagtccggtgcgtacaccggtgaagtttctgcaagcatgctggcaaagttgaactgctcttgggttgtcgttggacactccgagcgccgcgagtaccacaacgagtctgatgagttggttgctgcgaaggcaaaggcagctctgtccaacggcatcagcccgatcgtctgcgttggtgagccactggaaatccgtgaagctggcacccacgttgagtacgtcgtcgagcagacccgtaagtcccttgctggcctggatgctgctgagctggccaacaccgttatcgcgtatgagccagtgtgggctatcggcaccggtaaggttgcttccgcagctgacgctcaggaagtgtgcaaggctatccgcggtctgatcgtggagcttgcaggcgacgaggtcgctgagggcctgcgtattctttacggtggttctgttaaggcagaaaccgtcgctgagatcgtcggtcagcctgacgtcgacggcggacttgtcggtggcgcttccctcgacggtgaagcattcgccaagctggctgccaacgctgcgagcgttgcttaa

SEQ ID NO: 29 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 30 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 31 - protein, 461 aa, Corynebacterium glutamicum
MSDTPTSALITTVNRSFDGFDLEEVAADLGVRLTYLPDEELEVSKVLAADLLAEGPALIIGVGNTFFDAQVAAALGVPVLLLVDKQGKHVALARTQVNNAGAVVAAAFTAEQEPMPDKLRKAVRNHSNLEPVMSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 32 - protein, 397 aa, Corynebacterium glutamicum
MALALVLNSGSSSIKFQLVNPENSAIDEPYVSGLVEQIGEPNGRIVLKIEGEKYTLETPIADHSEGLNLAFDLMDQHNCGPSQLEITAVGHRVVHGGILFSAPELITDEIVEMIRDLIPLAPLHNPANVDGIDVARKILPDVPHVAVFDTGFFHSLPPAAALYAINKDVAAEHGIRRYGFHGTSHEFVSKRVVEILEKPTEDINTITFHLGNGASMAAVQGGRAVDTSMGMTPLAGLVMGTRSGDIDPGIVFHLSRTAGMSIDEIDNLLNKKSGVKGLSGVNDFRELREMIDNNDQDAWSAYNIYIHQLRRYLGSYMVALGRVDTIVFTAGVGENAQFVREDALAGLEMYGIEIDPERNALPNDGPRLISTDASKVKVFVIPTNEELAIARYAVKFA

SEQ ID NO: 33 - protein, 502 aa, Corynebacterium glutamicum
MSDRIASEKLRSKLMSADEAAQFVNHGDKVGFSGFTGAGYPKALPTAIANRAKEAHGAGNDYAIDLFTGASTAPDCDGVLAEADAIRWRMPYASDPIMRNKINSGSMGYSDIHLSHSGQQVEEGFFGQLNVAVIEITRITEEGYIIPSSSVGNNVEWLNAAEKVILEVNSWQSEDLEGMHDIWSVPALPNRIAVPINKPGDRIGKTYIEFDTDKVVAVVETNTADRNAPFKPVDDISKKIAGNFLDFLESEVAAGRLSYDGYIMQSGVGNVPNAVMAGLLESKFENIQAYTEVIQDGMVDLIDAGKMTVASATSFSLSPEYAEKMNNEAKRYRESIILRPQQISNHPEVIRRVGLIATNGLIEADIYGNVNSTNVSGSRVMNGIGGSGDFTRNGYISSFITPSEAKGGAISAIVPFASHIDHTEHDVMVVISEYGYADLRGLAPRERVAKMIGLAHPDYRPLLEEYYARATSGDNKYMQTPHDLATAFDFHINLAKNGSMKA

SEQ ID NO: 34 - protein, 1140 aa, Corynebacterium glutamicum
MSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS

SEQ ID NO: 35 - DNA, 43 bp, Artificial Sequence
  Synthetic ldhA_5′_HindIII
catgattacgccaagcttgagagcccaccacattgcgatttcc

SEQ ID NO: 36 - DNA, 42 bp, Artificial Sequence
  Synthetic ldhA_up_3′_XhoI
tcgaaactcgagtttcgatcccacttcctgatttccctaacc

SEQ ID NO: 37 - DNA, 39 bp, Artificial Sequence
  Synthetic ldhA_dn_5′_XhoI
tcgaaactcgagtaaatctttggcgcctagttggcgacg

SEQ ID NO: 38 - DNA, 46 bp, Artificial Sequence
  Synthetic ldhA_3′_EcoRI
acgacggccagtgaattcgacgacatctgagggtggataaagtggg

SEQ ID NO: 39 - DNA, 42 bp, Artificial Sequence
  Synthetic poxB 5′ H3
catgattacgccaagctttcagcgtgggtcgggttctttgag

SEQ ID NO: 40 - DNA, 32 bp, Artificial Sequence
  Synthetic DpoxB_up 3′
aatcatcatctgaactcctcaacgttatggct

SEQ ID NO: 41 - DNA, 37 bp, Artificial Sequence
  Synthetic DpoxB_dn 5′
ggagttcagatgatgattgatacacctgctgttctca

SEQ ID NO: 42 - DNA, 44 bp, Artificial Sequence
  Synthetic poxB 3′ E1
acgacggccagtgaattcatgtcccgaatccacttcaatcagag

SEQ ID NO: 43 - DNA, 44 bp, Artificial Sequence
  Synthetic pta 5′ H3
catgattacgccaagcttccctccatgatacgtggtaagtgcag

SEQ ID NO: 44 - DNA, 43 bp, Artificial Sequence
  Synthetic Dpta_up_R1 3′
gttccctgttaatgtaaccagctgaggtcggtgtgtcagacat

SEQ ID NO: 45 - DNA, 48 bp, Artificial Sequence
  Synthetic DackA_dn_R1 5′
ttacattaacagggaaccggaagagttagctatcgctaggtacgcggt

SEQ ID NO: 46 - DNA, 40 bp, Artificial Sequence
  Synthetic ackA 3′ Xb
acccggggatcctctagagggctgatgtgatttctgcggg

SEQ ID NO: 47 - DNA, 41 bp, Artificial Sequence
  Synthetic actA 5′ Xb
ggtggcggccgctctagaggtctgagctttattcctgggct

SEQ ID NO: 48 - DNA, 36 bp, Artificial Sequence
  Synthetic DactA_up_R4 3′
tctggatagaagcatctaagccagcgccggtgaagc

SEQ ID NO: 49 - DNA, 46 bp, Artificial Sequence
  Synthetic DactA_dn_R4 5′
agatgcttctatccagagctccggtgacaacaagtacatgcagacc

SEQ ID NO: 50 - DNA, 39 bp, Artificial Sequence
  Synthetic actA 3′ H3
gacggtatcgataagcttcgtacgatgcttgagcggtat

SEQ ID NO: 51 - DNA, 19 bp, Artificial Sequence
  Synthetic poxB_up_for
ggctgaaaccaaaccagac

SEQ ID NO: 52 - DNA, 22 bp, Artificial Sequence
  Synthetic poxB_dn_rev
ctgcatgatcggttagatacag

SEQ ID NO: 53 - DNA, 18 bp, Artificial Sequence
  Synthetic pta_up_for
gcgtggaattgagatcgg

SEQ ID NO: 54 - DNA, 18 bp, Artificial Sequence
  Synthetic ackA_dn_rev
cagagcgatttgtggtgg

SEQ ID NO: 55 - DNA, 20 bp, Artificial Sequence
  Synthetic actA_up_for
tgaagcaatggtgtgaactg

SEQ ID NO: 56 - DNA, 19 bp, Artificial Sequence
  Synthetic actA_dn_rev
gctaccaaacactagcctg

SEQ ID NO: 57 - DNA, 46 bp, Artificial Sequence
  Synthetic MD-616
aaagtgtaaagcctgggaacaacaagacccatcatagtttgccccc

SEQ ID NO: 58 - DNA, 36 bp, Artificial Sequence
  Synthetic MD-618
gttcttctaatcagaattggttaattggttgtaaca

SEQ ID NO: 59 - DNA, 40 bp, Artificial Sequence
  Synthetic MD-615
gcgtaatagcgaagaggggcgtttttccataggctccgcc

SEQ ID NO: 60 - DNA, 40 bp, Artificial Sequence
  Synthetic MD-617
gttcaatcataacaccccttgtattactgtttatgtaagc

SEQ ID NO: 61 - DNA, 31 bp, Artificial Sequence
  Synthetic MD-619
gggtgttatgattgaacaagatggattgcac

SEQ ID NO: 62 - DNA, 39 bp, Artificial Sequence
  Synthetic MD-620
attctgattagaagaactcgtcaagaaggcgatagaagg

SEQ ID NO: 63 - DNA, 17 bp, Artificial Sequence
  Synthetic LacZa-NR
cctcttcgctattacgc

SEQ ID NO: 64 - DNA, 21 bp, Artificial Sequence
  Synthetic MD-404
cccaggctttacactttatgc

SEQ ID NO: 65 - DNA, 47 bp, Artificial Sequence
  Synthetic MD-627
gccaccgcggtggagctcatttagcggatgattctcgttcaacttcg

SEQ ID NO: 66 - DNA, 32 bp, Artificial Sequence
  Synthetic MD-628
ttttatttgcaaaaacggccgaaaccatccct

SEQ ID NO: 67 - DNA, 40 bp, Artificial Sequence
  Synthetic MD-629
ccgtttttgcaaataaaacgaaaggctcagtcgaaagact

SEQ ID NO: 68 - DNA, 43 bp, Artificial Sequence
  Synthetic MD-630
gaacaaaagctggagctaccgtatctgtggggggatggcttgt

SEQ ID NO: 69 - DNA, 36 bp, Artificial Sequence
  Synthetic Tuf-F
ctatagggcgaattgggatcacagtaggcgcgtagg

SEQ ID NO: 70 - DNA, 56 bp, Artificial Sequence
  Synthetic Tuf-R
gacctcgagggggggcccggtaccggttgtcctcctttgggtggctacgactttcg

SEQ ID NO: 71 - DNA, 25 bp, Artificial Sequence
  Synthetic pyc-F1
gctctagattgagcacaccgtgact

SEQ ID NO: 72 - DNA, 19 bp, Artificial Sequence
  Synthetic pyc-R1
ctgaaggaggtgcgagtga

SEQ ID NO: 73 - DNA, 19 bp, Artificial Sequence
  Synthetic pyc-F2
tcactcgcacctccttcag

SEQ ID NO: 74 - DNA, 31 bp, Artificial Sequence
  Synthetic pyc-R2
gctctagagaagcagcatctgaatgtttaca

SEQ ID NO: 75 - DNA, 34 bp, Artificial Sequence
  Synthetic primer P246
ggattacgccaagcttcccgtaagtcccttgctg

SEQ ID NO: 76 - DNA, 37 bp, Artificial Sequence
  Synthetic primer P321
actgctcgagaactactttaaacactctttcacattg

SEQ ID NO: 77 - DNA, 32 bp, Artificial Sequence
  Synthetic primer P322
taaagtagttctcgagcagtaggcgcgtaggg

SEQ ID NO: 78 - DNA, 34 bp, Artificial Sequence
  Synthetic primer P323
aatcagtcatatgtatatctccttcctcgaggtg

SEQ ID NO: 79 - DNA, 34 bp, Artificial Sequence
  Synthetic primer P324
agatatacatatgactgattttttacgcgatgac

SEQ ID NO: 80 - DNA, 35 bp, Artificial Sequence
  Synthetic primer P325
acggccagtgaattcccaacattgccctcattgag

SEQ ID NO: 81 - DNA, 18 bp, Artificial Sequence
  Synthetic primer P250
ggcacccacgttgagtac

SEQ ID NO: 82 - DNA, 19 bp, Artificial Sequence
  Synthetic primer P326
cctcagcattgcgcagcac

SEQ ID NO: 83 - DNA, 32 bp, Artificial Sequence
  Synthetic primer P232
gattacgccaagcttggccactagcggagtgc

SEQ ID NO: 84 - DNA, 42 bp, Artificial Sequence
  Synthetic primer P242
gcgcctactgctcgagtacttctccagattttgtgtcattcg

SEQ ID NO: 85 - DNA, 30 bp, Artificial Sequence
  Synthetic primer P243
agtactcgagcagtaggcgcgtagggtaag

SEQ ID NO: 86 - DNA, 35 bp, Artificial Sequence
  Synthetic primer P244
cagtagtcatatgtatatctccttcctcgaggtgg

SEQ ID NO: 87 - DNA, 32 bp, Artificial Sequence
  Synthetic primer P245
agatatacatatgactactgctgcaatcaggg

SEQ ID NO: 88 - DNA, 34 bp, Artificial Sequence
  Synthetic primer P288
acggccagtgaattccatggaaccagcgtaatgc

SEQ ID NO: 89 - DNA, 36 bp, Artificial Sequence
  Synthetic primer P298
gatccgtcatatgtatatctccttcctcgaggtggc

SEQ ID NO: 90 - DNA, 34 bp, Artificial Sequence
  Synthetic primer P299
agatatacatatgacggatcttaaccagttgacg

SEQ ID NO: 91 - DNA, 31 bp, Artificial Sequence
  Synthetic primer P300
cagtcgtaggttaggcttttggtccggcagc

SEQ ID NO: 92 - DNA, 28 bp, Artificial Sequence
  Synthetic primer P301
aaaagcctaacctacgactgggagcacg

SEQ ID NO: 93 - DNA, 38 bp, Artificial Sequence
  Synthetic primer P302
acggccagtgaattcttaagcgtgagctgctgaaatgc

SEQ ID NO: 94 - DNA, 18 bp, Artificial Sequence
  Synthetic primer P303
ggtgtgtactgacagtgg

SEQ ID NO: 95 - DNA, 18 bp, Artificial Sequence
  Synthetic primer P304
ccgagaattcgcggtaag

SEQ ID NO: 96 - DNA, 34 bp, Artificial Sequence
  Synthetic primer ptsG_up_F
tgattacgccaagctttgcggggtgttttgttgt

SEQ ID NO: 97 - DNA, 33 bp, Artificial Sequence
  Synthetic primer ptsG_up_R
tcttgccgttgacctcagcgcctaccacttaat

SEQ ID NO: 98 - DNA, 38 bp, Artificial Sequence
  Synthetic primer ptsG_dn_F
aagtggtaggcgctgaggtcaacggcaagaacgagtaa

SEQ ID NO: 99 - DNA, 35 bp, Artificial Sequence
  Synthetic primer ptsG_dn_R
cagcgtgaagctagcggcgatacgagggtgggtaa

SEQ ID NO: 100 - DNA, 22 bp, Artificial Sequence
  Synthetic primer ptsG_C_F
tttttaaccttcacggtttggg

SEQ ID NO: 101 - DNA, 21 bp, Artificial Sequence
  Synthetic primer ptsG_C_R
aatcgacgagctgactcttcg

SEQ ID NO: 102 - protein, 919 aa, Corynebacterium glutamicum
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 103 - DNA, 2760 bp, Corynebacterium glutamicum
atgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaagcttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaagcttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttcacctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcgctgcgcaactccggctag

SEQ ID NO: 104 - protein, 538 aa, Mannheimia succiniciproducens
MTDLNQLTQELGALGIHDVQEVVYNPSYELLFAEETKPGLEGYEKGTVTNQGAVAVNTGIFTGRSPKDKYIVLDDKTKDTVWWTSEKVKNDNKPMSQDTWNSLKGLVADQLSGKRLFVVDAFCGANKDTRLAVRVVTEVAWQAHFVTNMFIRPSAEELKGFKPDFVVMNGAKCTNPNWKEQGLNSENFVAFNITEGVQLIGGTWYGGEMKKGMFSMMNYFLPLRGIASMHCSANVGKDGDTAIFFGLSGTGKTTLSTDPKRQLIGDDEHGWDDEGVFNFEGGCYAKTINLSAENEPDIYGAIKRDALLENVVVLDNGDVDYADGSKTENTRVSYPIYHIQNIVKPVSKAGPATKVIFLSADAFGVLPPVSKLTPEQTKYYFLSGFTAKLAGTERGITEPTPTFSACFGAAFLSLHPTQYAEVLVKRMQESGAEAYLVNTGWNGTGKRISIKDTRGIIDAILDGSIDKAEMGSLPIFDFSIPKALPGVNPAILDPRDTYADKAQWEEKAQDLAGRFVKNFEKYTGTAEGQALVAAGPKA

SEQ ID NO: 105 - DNA, 1617 bp, Mannheimia succiniciproducens
atgacggatcttaaccagttgacgcaggagttgggtgcgctgggcattcacgatgtccaggaggtggtttacaacccatcctacgaactgctgtttgcggaggaaaccaagccgggtcttgaaggatacgaaaaaggaacggtaactaaccaaggagcagtggcagtgaacaccggaatcttcaccggacggtcccctaaggacaaatacatcgtcctggacgacaagaccaaagatacggtctggtggacctcggagaaggtcaaaaacgataataaacccatgtcccaggacacgtggaacagcttgaagggcctggtcgcagaccaactttcaggtaagcgccttttcgtggtggatgcgttctgcggcgcaaacaaggacacccgcttggctgtccgcgtcgttactgaagtcgcatggcaggcacattttgtcaccaatatgttcatccgtccgtcggcggaagagttgaaagggttcaaacctgattttgttgtcatgaacggggccaagtgtaccaaccctaactggaaggagcaaggtctgaactccgagaacttcgtggcgttcaatatcactgagggcgttcagctcattgggggaacctggtatggtggggaaatgaagaagggtatgttctccatgatgaactacttcttgccgctgcgtgggattgcgtccatgcactgctcggcaaatgttggcaaggacggtgataccgcgatctttttcggccttagcggtacaggtaagaccaccttgtccaccgacccgaaacggcagttgattggtgatgacgagcacggttgggacgacgaaggcgtgtttaacttcgagggtggctgctatgcaaagactatcaacctctcagcggagaatgagcccgatatttacggcgcaatcaagcgcgatgcacttctggagaatgttgtcgttcttgacaatggcgacgttgactatgcagatggatctaagaccgagaacacccgcgtgagctaccccatctatcatatccaaaacatcgtaaaaccagtgtccaaggcaggcccggctactaaggtcatcttcctttcagccgatgcttttggcgtactgccacccgtgagcaagctcaccccagaacagaccaaatactacttcctctctggctttaccgccaaactcgctggtacagaacgaggtatcacagagccaaccccaaccttctctgcctgtttcggcgctgccttcctgtctctccaccctactcaatatgctgaggttctcgtgaaacgaatgcaggaatccggcgctgaagcatacctggttaataccggatggaacggcacgggcaagcgcatctctatcaaggatacccgtggtatcattgacgccatcctcgacggttctattgataaggctgaaatgggctcgctgccgattttcgatttttccatcccaaaggcactgccaggcgtgaaccctgccattctcgatcctcgtgatacttacgctgacaaggctcagtgggaggaaaaggcccaggatctcgctggacgcttcgtaaaaaacttcgaaaagtacactggcacagccgaaggccaggctctggttgctgccggaccaaaagcctaa

(19) United States
(12) Patent Application Publication
(10) Pub. No.: US 2015/0329883 A1
(43) Pub. Date: Nov. 19, 2015

(54) GENETICALLY ENGINEERED BACTERIAL CELL AND METHOD OF PRODUCING SUCCINIC ACID USING THE SAME

(71) Applicant: Samsung Electronics Co., Ltd., Suwon-si (KR)

(72) Inventors: Soonchun Chung, Seoul (KR); Jieun Kim, Suwon-si (KR); Joonsong Park, Seoul (KR); Jiae Yun, Hwaseong-si (KR); Jinhwan Park, Suwon-si (KR); Kwangmyung Cho, Seongnam-si (KR)

(21) Appl. No.: 14/716,408

(22) Filed: May 19, 2015

(30) Foreign Application Priority Data
May 19, 2014 (KR) 10-2014-0059969

(51) Int. Cl. C12P 7/46; C12N 9/16; C12N 9/10; C12N 9/12; C12N 9/04; C12N 9/02

(52) U.S. Cl. CPC C12P 7/46; C12N 9/0006; C12N 9/0008; C12N 9/1029; C12N 9/1217; C12N 9/16; C12N 9/13; C12Y 101/01027; C12Y 102/05001; C12Y 203/01008; C12Y 207/02001; C12Y 208/03; C12Y 301/02001

(57) ABSTRACT

Provided are a genetically engineered bacterial cell and a method of producing succinic acid by using the cell.

### RELATED APPLICATION

This application claims the benefit of Korean Patent Application No. 10-2014-0059969, filed on May 19, 2014, in the Korean Intellectual Property Office, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: One 104,058 byte ASCII (Text) file named “719830_ST25.TXT” created May 7, 2015.

### BACKGROUND

1. Field

The present disclosure relates to a genetically engineered bacterial cell and a method of producing succinic acid using the cell.

2. Description of the Related Art

A microorganism of the genus Corynebacterium is a gram-positive strain which is widely used to produce amino acids, such as glutamate, lysine, and threonine. Corynebacterium glutamicum grows under relatively simple culture conditions, has a stable genetic structure, and does not have a malignant influence on the environment, thus may be used as an industrial strain.

Corynebacterium glutamicum is an aerobe, and its growth ceases under anaerobic conditions or when oxygen supply is stopped. Under anaerobic conditions, metabolic processes of Corynebacterium glutamicum other than those necessary in producing minimum energy for survival are ceased, and lactic acid, acetic acid, or succinic acid are produced and released from Corynebacterium glutamicum.

A tricarboxylic acid (TCA) cycle is a metabolic pathway producing energy and intermediates in biospecies. The intermediates of the TCA cycle are synthesized into useful metabolites through series of metabolic pathways in a cell. Succinic acid is a decarboxylic acid that is used as a raw material of biodegradable polymers, medicine, food, or cosmetics. Most of succinic acid for industrial use is synthesized from n-butane and acetylene which are derived from petroleum or liquefied natural gas. Only a small quantity of succinic acid used for particular purposes, such as foods and medicines, is produced by microbial fermentation.

In general, a chemical synthesis process discharges a large amount of hazardous materials and uses a fossil fuel-derived raw material, which is a highly exhaustible resource, as a base material. Therefore, even when a conventional method is used, a microorganism capable of efficiently producing succinic acid and a method of producing succinic acid are needed.

### SUMMARY

Provided is a genetically engineered bacterial cell. The genetically engineered bacterial cell comprises increased activity of an expression product of a gene having about 95% or more sequence identity to at least one of Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, or Ncgl1524, wherein the activity of the expression product is increased in comparison to a non-engineered bacterial cell of the same type; and increased glucose consumption rate, increased succinic acid productivity, or both as compared to a non-engineered bacterial cell of the same type.

Also provided is a method of producing succinic acid by culturing the genetically engineered bacterial cell and collecting succinic acid from the culture.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a cleavage map of a pGSK+ vector;

FIG. 2 is a cleavage map of a pGST1 vector;

FIG. 3 is a cleavage map of a pGS-EX4 vector;

FIG. 4 is a graph illustrating succinic productivity and yield of a Corynebacterium glutamicum S003 strain according to a succinic acid concentration;

FIG. 5 is a graph illustrating glucose consumption rate of a Corynebacterium glutamicum S003 strain according to a succinic acid concentration;

FIG. 6 is a graph illustrating a glycolysis pathway in the presence of 0 M or 0.25 M of succinic acid and a glucose consumption rate of a recombination strain in which TCA-related genes (pgk, mdh, fda, and tpiA) are overexpressed;

FIG. 7 is a graph illustrating a glucose consumption rate of a recombination strain in which transcription regulator genes (NCgl0275, NCgl0368, NCgl1853, NCgl1855, NCgl2199, NCgl2359, NCgl2404, NCgl2425, and NCgl2988) are overexpressed in the presence of 0 M or 0.25 M of succinic acid;

FIG. 8 is a graph illustrating an amount of succinic acid production of a recombination strain in which transcription regulator genes (NCgl0275, NCgl0368, NCgl1853, NCgl1855, NCgl2199, NCgl2359, NCgl2404, NCgl2425, and NCgl2988) are overexpressed in the presence of 0 M or 0.25 M of succinic acid;

FIG. 9 is a graph illustrating a glucose consumption rate of a recombination Corynebacterium strain in which a NCgl0275 gene is overexpressed in the presence of various concentrations of succinic acid;

FIG. 10 is a graph illustrating results of fermentation using a recombined Corynebacterium glutamicum S003 strain;

FIG. 11 is a graph illustrating results of fermentation of a recombined Corynebacterium glutamicum S003 strain in which a NCgl0275 gene is overexpressed;

FIG. 12 is a graph illustrating a succinic acid productivity and a yield of the recombined Corynebacterium strain; and

FIG. 13 is a graph illustrating results of fermentation of a recombined Corynebacterium S071 strain in which a NCgl0275 gene is overexpressed.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

As used herein, the term “increase in activity” of an expression product may refer to a sufficient increase in the amount thereof to show activity thereof and may also refer to an activity level of a cell or an isolated expression product that is higher than that of a comparable cell of the same type or an original expression product. In other words, the activity of the expression product may be increased by about 5% or more, about 10% or more, about 15% or more, about 20% or more, about 30% or more, about 50% or more, about 60% or more, about 70% or more, or about 100%, compared to the same biochemical activity of a non-engineered expression product. Also, an activity of a particular expression product in the corresponding cell may be increased by about 5% or more, about 10% or more, about 15% or more, about 20% or more, about 30% or more, about 50% or more, about 60% or more, about 70% or more, about 100%, about 200% or about 300%, compared to the same activity of an expression product in a non-engineered cell. The expression product having increased activity may be identified by using a method known in the art.

As used herein, the term “expression product” includes a material that is produced from a gene by at least one of transcription and translation. The expression product may be mRNA, protein, or a combination thereof. The protein may be an enzyme.

The increased activity of the expression product may occur due to an increased expression or an increased specific activity. The increased expression may occur by introducing a polynucleotide encoding the expression product into a cell, increasing a copy number of the polynucleotide in the cell, or mutating (modifying) a regulatory region of the polynucleotide. A polynucleotide that is introduced or present in an increased copy number may be an endogenous gene or an exogenous gene. The endogenous gene refers to a gene that exists in a genetic material included in a microorganism. The exogenous gene refers to a gene that is introduced to a cell from the outside, wherein the introduced gene may be homologous or heterologous with respect to the host cell genome.

The expression “increased copy number” may include a copy number increase by an introduction or amplification of the gene. The expression “increased copy number” may also include a copy number increase by genetically manipulating a cell that does not have a gene so as to have the gene in the cell. The introduction of the gene may occur by using a vehicle such as a vector. The introduction may be a transient introduction, in which the gene is not integrated into the genome, or an integration into the genome. The introduction may, for example, occur by introducing a vector inserted with a polynucleotide encoding a desired polypeptide into the cell and then replicating the vector in the cell or integrating the polynucleotide into the genome of the cell and then replicating the polynucleotide together with the replication of the genome.

The term “gene” as used herein refers to a nucleic acid fragment from which the expression product, such as mRNA or protein, may be produced by at least one of transcription and translation and may include a regulatory sequence such as a 5′-non-coding sequence and a 3′-non-coding sequence in addition to a coding region.

The term “heterologous” as used herein refers to foreign matter that is not native to the cell.

The term “excretion” as used herein refers to a movement of a material from a cell interior to a periplasmic space or an extracellular environment.

The terms “cell”, “strain”, or “microorganism” as used herein may be interchangeably used and may include bacteria, yeast, fungi, or the like.

The reduced activity of the enzyme may be due to deletion or disruption of the gene encoding the enzyme. The “deletion” or the “disruption” of the gene refers to mutation of some or the whole gene, or regulatory regions including promoter or a terminator region thereof, such that the gene may not be expressed, have reduced expression, or show no activity or reduced activity of the enzyme, even when the gene is expressed. The mutation includes addition, substitution, insertion, or conversion of at least one base of the gene. The deletion or the disruption of the gene may be achieved by genetic manipulation such as homologous recombination, mutagenesis, or molecular evolution. When a cell includes a plurality of the same genes or two or more of different paralogs, one or more genes may be removed or disrupted.

As used herein, the term “a sequence identity” of nucleic acid or polypeptide according to an embodiment of the present disclosure refers to the extent of identity between bases or amino acid residues of sequences after aligning the sequences such that they maximally match in certain comparative regions. The sequence identity is a value calculated by optimally aligning two sequences at certain comparative regions, wherein portions of the sequences at the certain comparative regions may be added or deleted, compared to reference sequences. A percentage of sequence identity may be calculated by, for example, comparing two optimally aligned sequences in the entire comparative region, determining the number of locations in which the same amino acids or nucleic acids appear to obtain the number of matched locations, dividing the number of matched locations by the total number of locations in the comparative region (that is, the size of the range), and multiplying by 100 to calculate the percentage of the sequence identity. The percentage of the sequence identity may be calculated by using a known sequence comparison program, and examples of such program include BLASTN (NCBI), CLC Main Workbench (CLC bio), and MegAlign™ (DNASTAR Inc).

Various levels of sequence identity may be used to identify various types of polypeptides or polynucleotides having the same or similar functions. For example, a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100% may be used.

As used herein, the term “non-engineered cell” may denote that at least one gene selected from the group consisting of Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, and Ncgl1524 is not genetically engineered for an activity of the expression product to be increased. The term “non-engineered cell” may also be a parent strain that is used to engineer a cell which does not contain a particular modification. As used herein, the term “genetic engineering” and similar terms denotes artificially modifying a component or a structure of a gene material of a cell. The non-engineered cell may be a parent strain that is used to engineer at least one gene selected from the group consisting of Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, and Ncgl1524 so that an activity of the expression product increases.

According to one aspect of the present disclosure, provided is a bacterial cell that is genetically engineered to overcome inhibition of succinic acid production due to succinic acid by increasing activity of a gene of which expression is specifically suppressed by succinic acid. The bacterial cell may have at least one characteristic selected from the group consisting of an increase in succinic acid production, an increase in succinic acid production yield, and a glucose consumption rate.

According to one aspect of the present disclosure, provided is a genetically engineered bacterial cell having increased activity of an expression product of a gene having about 95% or more sequence identity to at least one of Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, or Ncgl1524, wherein the activity is increased in comparison to a non-engineered bacterial cell of the same type; and increased glucose consumption rate, increased succinic acid productivity, or both as compared to a non-engineered bacterial cell of the same type.

When the bacterial cell is cultured in the presence of succinic acid, the at least one gene may be selected from genes having a decreased amount of their expressions compared to the case when the succinic acid is not present. The genes may have characteristics shown in Table 1.

TABLE 1

| | Ncgl No. | | | | | |
|---|---|---|---|---|---|---|
| | (SEQ ID | | Fold of | | Length of | |
| Expected function | NO.) | Gene name | reduction* | p Value | amino acid | Gene product |
| Transcriptional | Ncgl1853 | nrdR | 4.24 | 0.003 | 150 | Potential regulator |
| regulator | (SEQ ID | | | | | |
| | NO: 15) | | | | | |
| | Ncgl1855 | lexA | 2.44 | 0.004 | 253 | Potential lexA repressor |
| | (SEQ ID | | | | | |
| | NO: 16) | | | | | |
| | Ncgl2988 | parB | 1.91 | 0.016 | 379 | Predicted transcriptional |
| | (SEQ ID | | | | | regulator related to cell |
| | NO: 17) | | | | | division chromosome |
| | | | | | | partitioning protein, ParB |
| | | | | | | family |
| | Ncgl2425 | | 1.74 | 0.017 | 164 | Bacteria regulatory protein, |
| | (SEQ ID | | | | | MarR family |
| | NO: 18) | | | | | |
| | Ncgl2404 | | 1.73 | 0.013 | 212 | Bacteria regulatory protein, |
| | (SEQ ID | | | | | tetR family |
| | NO: 19) | | | | | |
| | Ncgl0368 | | 1.60 | 0.015 | 202 | TetR-family transcriptional |
| | (SEQ ID | | | | | regulator |
| | NO: 20) | | | | | |
| | Ncgl2877 | | 1.57 | 0.071 | 192 | Transcriptional regulator |
| | (SEQ ID | | | | | PadR-like family |
| | NO: 21) | | | | | |
| | Ncgl0275 | whiB4 | 1.56 | 0.035 | 116 | Potential regulator protein |
| | (SEQ ID | | | | | (whiB-related) |
| | NO: 22) | | | | | |
| | Ncgl2359 | | 1.52 | 0.034 | 246 | Bacteria regulator protein, |
| | (SEQ ID | | | | | TetR family |
| | NO: 23) | | | | | |
| Glycolysis pathway | Ncgl1525 | pgk | 0.436 | 0.011 | 405 | phosphoplycerate kinase |
| and function related | (SEQ ID | | | | | |
| to TCA cycle | NO: 24) | | | | | |
| | Ncgl0976 | glpX | 1.910 | 0.010 | 335 | glpX-like protein |
| | (SEQ ID | | | | | |
| | NO: 25) | | | | | |
| | Ncgl2673 | fda | 1.820 | 0.040 | 344 | Fructose-bisphosphate |
| | (SEQ ID | | | | | aldolase |
| | NO: 26) | | | | | |
| | Ncgl2297 | mdh | 1.700 | 0.020 | 328 | malate dehydrogenase |
| | (SEQ ID | | | | | oxidoreductase protein |
| | NO: 27) | | | | | |
| | Ncgl1524 | tpi | 1.700 | 0.020 | 259 | triosephosphate isomerase |
| | (SEQ ID | | | | | |
| | NO: 28) | | | | | |

*A fold of reduction is obtained by comparing the expression of Corynebacterium glutamicum ATCC13032 as cultured under an anaerobic condition in the same manner as in Example 1 for 5 hours in the presence of 0.0625M of succinic acid with a control group, which is cultured without the succinic acid.

When the bacterial cell is cultured in the presence of succinic acid, the cell may have an increased glucose consumption rate, an increased succinc acid productivity, or both of the increased characteristics compared to that of a non-engineered cell.

A concentration of the succinic acid may be in, for example, about 1 uM to about 2 M, about 1 uM to about 1 M, about 10 uM to about 1 M, about 100 uM to about 1 M, about 1000 uM to about 1 M, about 1 mM to about 1 M, about 10 mM to about 1 M, about 100 mM to about 1 M, about 1 uM to about 0.8 M, about 10 uM to about 0.8 M, about 100 uM to about 0.8 M, about 1000 uM to about 0.8 M, about 1 mM to about 0.8 M, about 10 mM to about 0.8 M, about 100 mM to about 0.8 M, about 1 mM to about 10 M, about 1 mM to about 5 M, or about 1 mM to about 2 M.

The bacterial cell may have a succinic acid productivity under a microaerobic condition or an anaerobic condition. The microaerobic condition may denote a culturing condition in which a low level of oxygen dissolved in a culturing medium. The low level of oxygen denotes a level of oxygen lower than that in the air. The low level of oxygen may be about 0.1% to about 10%, about 1% to about 9%, about 2% to about 8%, about 3% to about 7%, or about 4% to about 6% of a saturated dissolved oxygen concentration in the air.

The bacterial cell may be Corynebacterium genus. The cell may be, for example, Corynebacterium glutamicum, Corynebacterium thermoaminogenes, Brevibacterium flavum, or Brevibacterium lactofermentum. The Corynebacterium glutamicum may be a Corynebacterium glutamicum l ATCC 13032 strain.

The increased amount or activity of the gene having a sequence identity of about 95% or more with the expression product of the at least one gene may be caused by an increased number of copies of the at least one gene or modification of an expression regulating (regulatory) sequence of the gene.

The increased number of copies may be caused by introduction of the gene from the outside of the cell to the inside (e.g., introduction of an exogenous gene) or by amplification of an intrinsic (internal or “endogenous”) gene.

The introduction may be mediated by a vehicle such as a vector. The introduction may be transient introduction where the gene is not combined into the genome or introduction inserted to the genome. The introduction may be performed by, for example, introducing the gene-inserted vector to the cell and then allowing the vector to be copied in the cell or combining the gene into the genome. The gene may be operably linked to a regulatory sequence involved in controlling the expression. The regulatory sequence may include a promoter, a 5′-non-coding sequence, a 3′-non-coding sequence, a transcription terminator sequence, an enhancer, or a combination thereof. The gene may be an endogenous gene or an exogenous gene. The regulatory sequence may be a sequence that encodes a motif which may influence the gene expression. The motif may be, for example, a secondary structure-stabilizing motif, an RNA destabilizing motif, a splice-activating motif, a polyadenylation motif, an adenine-rich sequence, or an endonuclease recognizing site.

The increased activity of the expression product of the at least one gene may be caused by mutation of the at least one gene. The mutation may be a substitution, insertion, addition, or conversion of at least one base in the gene.

The at least one gene may encode an expression product having a sequence identity of about 95% or more with at least one amino acid sequence selected from SEQ ID NOS: 1 to 14.

The at least one gene may have sequence identity of about 95% or more with at least one nucleotide sequence selected from SEQ ID NOS: 15 to 28.

In the bacterial cell, an L-lactate dehydrogenase gene, a pyruvate oxidase gene, a phosphotransacetylase gene, an acetate kinase gene, an acetate CoA transferase gene, or a combination thereof may be removed (deleted) or disrupted.

The L-lactate dehydrogenase (LDH) may catalyze conversion of lactate to pyruvate. The LDH may be an enzyme that is classified under EC.1.1.1.27. For example, the LDH may have an amino acid sequence of SEQ ID NO: 29. The LDH gene may encode an amino acid sequence of SEQ ID NO: 29.

The pyruvate oxidase (PoxB) may catalyze conversion of pyruvate to acetate. The PoxB may be an enzyme that is classified under EC.1.2.5.1. For example, the PoxB may have an amino acid sequence of SEQ ID NO: 30. The PoxB gene may encode an amino acid sequence of SEQ ID NO: 30.

The phosphotransacetylase (PTA) may catalyze conversion of acetyl-CoA to acetylphosphate. The PTA may be an enzyme that is classified under EC.2.3.1.8. For example, the PTA may have an amino acid sequence of SEQ ID NO: 31. The PTA gene may encode an amino acid sequence of SEQ ID NO: 31.

The acetate kinase (AckA) may catalyze conversion of acetate to acetyl phosphate. The AckA may be an enzyme that is classified under EC.2.7.2.1. For example, the AckA may have an amino acid sequence of SEQ ID NO: 32. The AckA gene may encode an amino acid sequence of SEQ ID NO: 32.

The acetate coenzyme A transferase (ActA) may catalyze conversion of acetyl-CoA to acetate and CoA (a reversible reaction). The ActA may be an enzyme that is classified under EC.3.1.2.1 or EC.2.8.3.-. For example, the ActA may have an amino acid sequence of SEQ ID NO: 33. The ActA gene may encode an amino acid sequence of SEQ ID NO: 33.

The bacterial cell may have increase in activity of a pyruvate carboxylase (PYC) catalyzing conversion of pyruvate to oxaloacetate. As used herein, the term “increase in activity” is as described herein. The increase in activity may be caused by introduction of a gene that encodes a PYC with an increased specific activity due to modification of the PYC. The modification may include substitution, addition, deletion, or a combination thereof of the PYC. The substitution may be replacement of 458^{th} proline of SEQ ID NO: 34 with serine, that is, a P458S substitution. The cell may have an activity that is increased by, for example, random mutation or genetic engineering.

The bacterial cell may have an increased activity of a phosphoenolpyruvate carboxylase (PEPC). The PEPC may catalyze conversion of phosphoenolpyruvate and bicarbonate to oxaloacetate. The PEPC may be an enzyme that is classified under EC.4.1.1.31. The PEPC may have an amino acid sequence of SEQ ID NO: 102. The PEPC gene may have a nucleotide sequence of SEQ ID NO: 103.

The bacterial cell may have an activity of a phosphoenolpyruvate carboxykinase (pck) that has an activity of converting PEP to OAA. The pck gene may be, for example, a pck gene of Mannheimia succiniciproducens. The pck may have an amino acid sequence of SEQ ID NO: 104. The pck gene may have a nucleotide sequence of SEQ ID NO: 105.

The bacterial cell may be a strain in which activity of a glucose-specific Ellpermease (ptsG) is decreased in comparison to a non-engineered cell.

According to another aspect of the present disclosure, provided is a method of producing succinic acid, the method including culturing the bacterial cell described above in a culture medium; and collecting succinic acid from the culture.

The culturing of the microorganism may be performed in a suitable medium under suitable culturing conditions known in the art. One of ordinary skill in the art may suitably change a culture medium and culturing conditions according to the microorganism selected. A culturing method may be batch culturing, continuous culturing, fed-batch culturing, or a combination thereof. The bacterial cell is as described herein.

The culture medium may include various carbon sources, nitrogen sources, and trace elements.

The carbon source may be, for example, carbohydrate such as glucose, sucrose, lactose, fructose, maltose, starch, or cellulose; fat such as soybean oil, sunflower oil, castor oil, or coconut oil; fatty acid such as palmitic acid, stearic acid, linoleic acid; alcohol such as glycerol or ethanol; organic acid such as acetic acid, and/or a combination thereof. For example, the culturing may be performed by having glucose as the carbon source. For example, the nitrogen source may be an organic nitrogen source such as peptone, yeast extract, beef stock, malt extract, corn steep liquor (CSL), or soybean flour, or an inorganic nitrogen source such as urea, ammonium sulfate, ammonium chloride, ammonium phosphate, ammonium carbonate, and ammonium nitrate, and/or a combination thereof. The culture medium is a supply source of phosphorus and may include, for example, potassium dihydrogen phosphate, dipotassium phosphate, and corresponding sodium-containing salt, and a metal salt such as magnesium sulfate or iron sulfate. Also, amino acid, vitamin, or a suitable precursor may be included in the culture medium. The culture medium or individual component may be added to a culture medium solution in a batch or continuous manner.

Also, pH of the culture medium solution may be adjusted by adding a compound such as ammonium hydroxide, potassium hydroxide, ammonia, phosphoric acid, and sulfuric acid to the culture medium solution by using a suitable method during the culturing process. Also, an antifoaming agent such as fatty acid polyglycol ester may be used during the culturing process to inhibit the generation of bubbles.

The cell may be cultured under an aerobic, microaerobic, or anaerobic condition. The microaerobic condition may denote a culturing condition in which a level of oxygen lower than that in the air is dissolved in a culturing medium. The low level of oxygen may be about 0.1% to about 10%, about 1% to about 9%, about 2% to about 8%, about 3% to about 7%, or about 4% to about 6% of a saturated dissolved oxygen concentration in the air. Also, the microaerobic condition may denote, for example, about 0.9 ppm to about 3.6 ppm of a dissolved oxygen concentration in the medium. The culturing temperature may be, for example, about 20° C. to about 45° C. or about 25° C. to about 40° C. The culturing time may be continued until a desired amount of the desired succinic acid is obtained.

The culturing may be performed in the presence of succinic acid at a concentration of about 1 uM to about 2M, about 1 uM to about 1 M, about 10 uM to about 1 M, about 100 uM to about 1 M, about 1000 uM to about 1 M, about 1 mM to about 1 M, about 10 mM to about 1 M, about 100 mM to about 1 M, about 1 uM to about 0.8 M, about 10 uM to about 0.8 M, about 100 uM to about 0.8 M, about 1000 uM to about 0.8 M, about 1 mM to about 0.8 M, about 10 mM to about 0.8 M, about 100 mM to about 0.8 M, about 1 mM to about 10 M, about 1 mM to about 5 M, or about 1 mM to about 2 M.

The culturing may be performed at a pH of about 2 to about 7.5, for example, pH of about 2 to about 6, pH of about 2 to about 5, pH of about 2 to about 4, pH of about 2 to about 3, pH of about 3 to about 7.5, pH of about 3 to about 6, pH of about 3 to about 5, or pH of about 3 to about 4. The pH may be achieved within the culture itself by accumulation of succinic acid without adding an appropriate buffer material from the outside or adding an outside material as the cultivation proceeds.

The collecting of succinc acid from the culture may be performed by a separation and purification method known in the art. The collecting may be performed by centrifugation, ion-exchange chromatography, filtration, precipitation, or a combination thereof. For example, the biomass may be removed by performing centrifugation, and the supernatant obtained therefrom may be separated through ion-exchange chromatography.

The genetically engineered bacterial cell according to an aspect of the present disclosure may be effectively used in production of metabolites as the cell has at least one of a high glucose consumption rate or a high succinic acid productivity.

According to another aspect of the present disclosure, succinic acid may be effectively produced by using a method of producing succinic acid.

Hereinafter, the present disclosure is described in greater detail with reference to embodiments. However, the embodiments are for illustrative purposes only and do not limit the scope of the present invention in any fashion.

Material and Method

Materials and methods described below are used in an example if not particularly mentioned.

1. Corynebacterium S003 Strain

A Corynebacterium S003 strain is a recombination strain from which lactate and acetate synthesis pathways are removed by having C. glutamicum (CGL) ATCC 13032 as a mother strain. The strain was prepared as follows.

(1) Preparation of Replacement Vector

An L-lactate dehydrogenase (ldh) gene, a pyruvate oxidase (poxB) gene, a phosphotransacetylase (pta) gene, an acetate kinase (ackA) gene, and an acetate CoA transferase (actA) gene of Corynebacterium glutamicum ATCC 13032 were inactivated by homologous recombination. A vector for the inactivation of the genes was a pK19 mobsacB (ATCC 87098) vector, and homologous sites for the recombination were obtained by amplification through PCR using a genomic DNA of CGL ATCC 13032 as a template.

The two homologous sites for deletion of the ldh gene are an upstream site and a downstream site of the gene. The two homologous sites were obtained by PCR amplification using a primer set of IdhA_{—}5′_HindIII (SEQ ID NO: 35) and IdhA_up_{—}3′Xhol (SEQ ID NO: 36) and a primer set of IdhA_dn_{—}5′Xhol (SEQ ID NO: 37) and IdhA_{—}3′EcoRI (SEQ ID NO: 38). The PCR amplification was performed by running 30 cycles of PCR, where each of the cycles included 30 seconds of denaturation at 95° C., 30 seconds of annealing at 95° C., and 30 seconds of elongation at 72° C. Hereinafter, all PCR amplifications were performed in the same manner. The amplified products thus obtained were cloned at locations of HindIII and EcoRI restriction enzymes of a pK19 mobsacB vector to prepare a pK19_Δldh vector.

The two homologous sites for deletion of the pox B gene are an upstream site and a downstream site of the gene. The two homologous sites were obtained by PCR amplification using a primer set of poxB 5′ H3 (SEQ ID NO: 39) and DpoxB_up 3′ (SEQ ID NO: 40) and a primer set of DpoxB_dn 5′ (SEQ ID NO: 41) and poxB 3′ E1 (SEQ ID NO: 42). The amplified products thus obtained were cloned at locations of HindIII and EcoRI restriction enzymes of a pK19 mobsacB vector to prepare a pK19_ΔpoxB vector.

The two homologous sites for deletion of the pta-ackA gene are an upstream site and a downstream site of the gene. The two homologous sites were obtained by PCR amplification using a primer set of pta 5′ H3 (SEQ ID NO: 43) and Dpta_up_R13′ (SEQ ID NO: 44) and a primer set of DackA_dn_R1 5′ (SEQ ID NO: 45) and ackA 3′ Xb (SEQ ID NO: 46). The amplified products thus obtained were cloned at locations of HindIII and EcoRI restriction enzymes of a pK19 mobsacB vector to prepare a pK19_Δpta_ackA vector.

The two homologous sites for deletion of the actA gene are an upstream site and a downstream site of the gene. The two homologous sites were obtained by PCR amplification using a primer set of actA 5′ Xb (SEQ ID NO: 47) and DactA_up_R4 3′ (SEQ ID NO: 48) and a primer set of DactA_dn_R4 5′ (SEQ ID NO: 49) and actA 3′ H3 (SEQ ID NO: 50). The amplified products thus obtained were cloned at locations of HindIII and EcoRI restriction enzymes of a pK19 mobsacB vector to prepare a pK19_ΔactA vector.

(2) Preparation of CGL (Δldh, ΔpoxB, Δpta-ackA, and ΔactA)

The replacement vectors were introduced to a C. glutamicum ATCC 13032 strain by electroporation. The introduced strain was smeared on an LBHIS agar plate containing 25 ug/ml of kanamycin and then cultured at a temperature of about 30° C. An LBHIS culture medium includes 25 g/L of Difco LB™ broth, 18.5 g/L of brain-heart infusion broth, 91 g/L of D-sorbitol, and 15 g/L or agar. Hereinafter, the composition of the LBHIS culture medium is as described above. The colony formed therefrom was smeared on a BHIS culture medium (pH 7.0) including 37 g/L of brain heart infusion powder and 91 g/L of D-sorbitol and then cultured at a temperature of 30° C., followed by smearing the culture on an LB/Suc10 agar plate, culturing at a temperature of 30° C., and selecting colonies in which double cross-over occurred. The LB/Suc10 agar plate includes 25 g/L of Difco LB™ broth, 15 g/L of agar, and 100 g/L of sucrose.

Genomic DNA was separated from the selected colonies, and then deletion of the genes was confirmed. PCR using a primer set of IdhA_{—}5′_HindIII and IdhA_{—}3′_EcoRI was performed to confirm deletion of the ldh gene, and PCR using a primer set of poxB_up_for (SEQ ID NO: 51) and poxB_dn_rev (SEQ ID NO: 52) was performed to confirm deletion of the poxB gene. Also, PCR using a primer set of pta_up_for (SEQ ID NO: 53) and ackA_dn_rev (SEQ ID NO: 54) was performed to confirm deletion of the pta-ackA gene, and PCR using a primer set of actA_up_for (SEQ ID NO: 55) and actA_dn_rev (SEQ ID NO: 56) was performed to confirm deletion of the ackA gene.

2. Preparation of pGEX_Ptuf::Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, and Ncgl1524 Vectors

14 genes were each introduced to a Xhol/BamHI site of a pGS_EX4 vector and thus a vector operably linked with a Ptuf promoter was prepared.

(1) Preparation of pGS_EX4 Vector

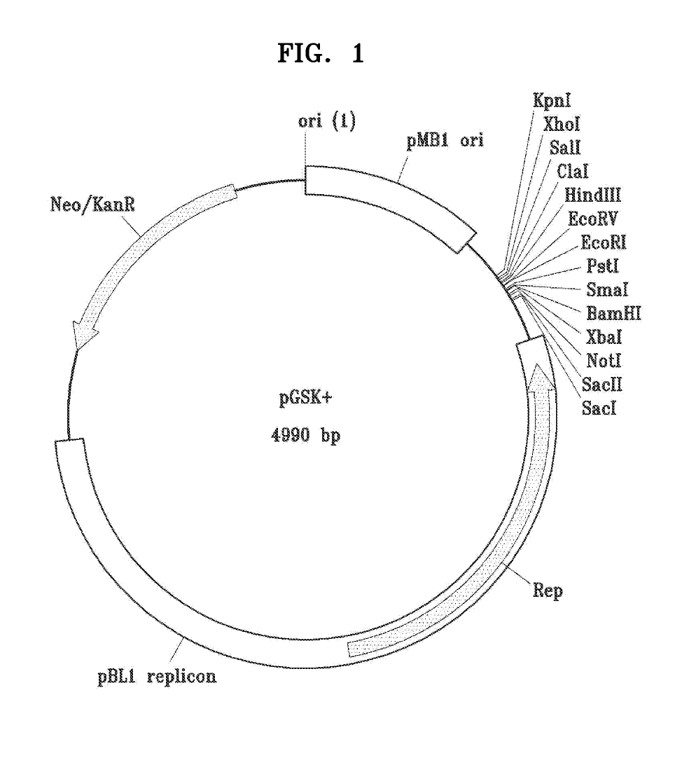

Four PCR products below were obtained by using a phusion high-fidelity DNA polymerase (New England Biolabs, cat. #M0530). PCR was performed by using pET2 (GenBank accession number: AJ885178.1), which is a vector for promoter screening of Corynebacterium glutamicum, as a template and using a primer set of MD-616 (SEQ ID NO: 57) and MD-618 (SEQ ID NO: 58) and a primer set of MD-615 (SEQ ID NO: 59) and MD-617 (SEQ ID NO: 60). Also, PCR was performed by using pEGFP-C1 (Clontech) as a template and using a primer set of MD-619 (SEQ ID NO: 61) and MD-620 (SEQ ID NO: 62), and PCR was performed by using pBluescriptII SK+ as a template and using a primer set of LacZa-NR (SEQ ID NO: 63) and MD-404 (SEQ ID NO: 64). Each of PCR products, 3010 bp, 854 bp, 809 bp, and 385 bp was cloned into a circular plasmid according to a method described in In-Fusion EcoDry PCR Cloning Kit (cat. #639690, available from Clontech). The cloned vector obtained therefrom was transformed into a One Shot TOP10 Chemically Competent Cell (cat. #C4040-06, a product of Invitrogen) and then cultured in 25 ug/L of a kanamycin-containing LB culture medium, followed by selection of growth colonies. Vectors were collected from the selected colonies to analyze a vector sequence by using a whole sequence analysis. The vector was named pGSK+ (FIG. 1). FIG. 1 is a cleavage map of a pGSK+ vector.

Also, 3′UTR of C. glutamicum gltA (NCgl0795) and a rho-independent terminator of E. coli rrnB were inserted to the pGSK+ vector in the following manner. A 108 bp PCR fragment of gltA 3′UTR was obtained from PCR using C. glutamicum (ATCC 13032) genomic DNA as a template and a primer set of MD-627 (SEQ ID NO: 65) and MD-628 (SEQ ID NO: 66). Also, a 292 bp PCR product of a rrnB transcription terminator was obtained from PCR using E. coli (MG1655) genomic DNA as a template and a primer set of MD-629 (SEQ ID NO: 67) and MD-630 (SEQ ID NO: 68).

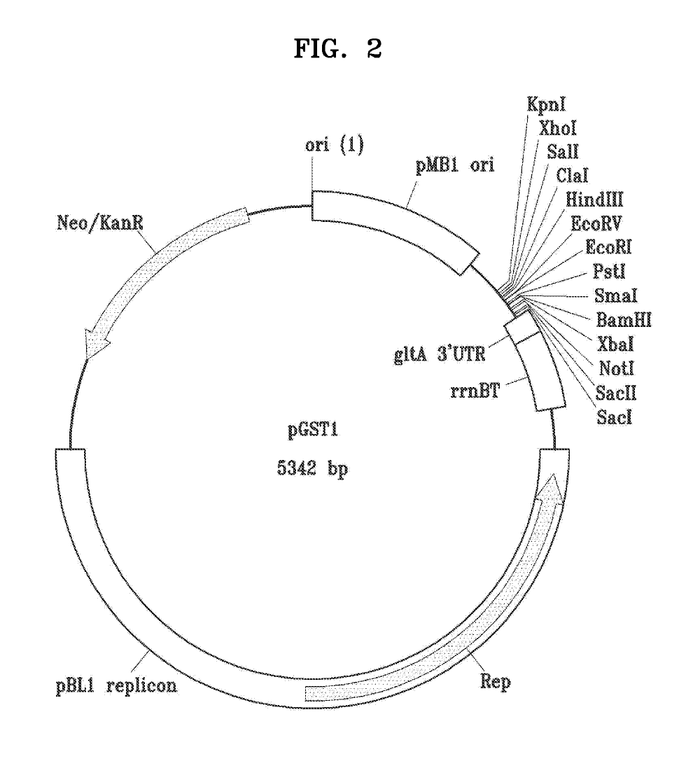

Two amplified fragments were inserted into pGSK+ cleaved by Sac! using In-Fusion EcoDry PCR Cloning Kit (cat. #639690, a product of Clontech). The cloned vector was introduced into One Shot TOP10 Chemically Competent Cell (cat. #C4040-06, a product of Invitrogen) and then cultured in 25 ug/L of kanamycin-containing LB culture medium, followed by selection of growth colonies therefrom. A vector was recovered from the selected colonies to analyze a vector sequence through a whole sequence analysis. The vector was named pGST1 (FIG. 2). FIG. 2 illustrates a cleavage map of a pGST1 vector.

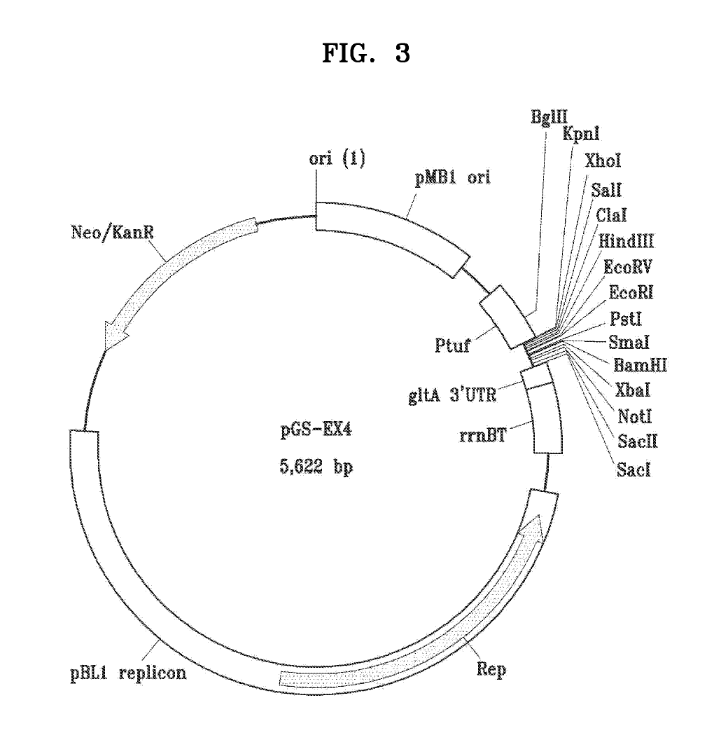

Also, a Ptuf fragment was obtained by using genomic DNA of C. glutamicum ATCC 13032 as a template and a primer set of Tuf-F (SEQ ID NO: 69) and Tuf-R (SEQ ID NO: 70). Ptuf is a promoter of a tuf gene (NCg10480) derived from Corynebacterium glutamicum. The obtained Ptuf fragment was cloned to a Kpnl site of the pGST1 vector by using In-Fusion® HD Cloning Kit (Clontech 639648) to obtain a pGS_EX4 vector (FIG. 3). FIG. 3 illustrates a cleavage map of a pGS-EX4 vector.

(2) Preparation of pGEX_Ptuf::Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, and Ncgl1524 Vector

Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, and Ncgl1524 genes of Corynebacterium glutamicum ATCC 13032 were amplified by PCR using primer sets each respectively having BamHI and Xhol at an end and genomic DNA of Corynebacterium glutamicum ATCC 13032 as a template. In order to express the genes in the presence of the tuf promoter of Corynebacterium glutamicum, the genes were cloned at the sites of BamHI and Xhol of the pGS_EX4 vector shown in FIG. 3 to obtain pGEX_Ptuf::Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, and Ncgl1524 vectors.

3. Corynebacterium glutamicum (Δldh, ΔpoxB, Δpta-ackA, ΔactA, pyc^{P458S})

Corynebacterium glutamicum (Δldh, ΔpoxB, Δpta-ackA, ΔactA, pyc^{P458S}) was prepared as follows.

A mutant (hereinafter, also referred to as ‘PYC^{P458S}’), in which 458^{th} proline of pyruvate carboxylase (SEQ ID NO: 34) of C. glutamicum ATCC 13032 is substituted with serine, was prepared.

The mutant was prepared by substituting codon CCG was substituted with TCG, wherein the codon CCG codes 458^{th} proline in a PYC amino acid sequence by using an overlap extension PCR method.

From a genomic DNA of CGL ATCC 13032, a PCR product was obtained by PCR amplification using a primer set of pyc-F1 (SEQ ID NO: 71) and pyc-R1 (SEQ ID NO: 72), and a PCR product was obtained by PCR amplification using a primer set of pyc-F2 (SEQ ID NO: 73) and pyc-R2 (SEQ ID NO: 74). A PCR product was obtained by PCR amplification using the two PCR products as a template and a primer set of pyc-F1 and pyc-R2. The amplification product obtained therefrom was cloned at a location of a XbaI restriction enzyme of a pK19mobsacB vector to prepare a pK19mobsacB-pyc* vector.

The pK19mobsacB-pyc* vector was introduced to CGL (Δldh, ΔpoxB, Δpta-ackA, ΔactA) of “1”. PCR was performed by using a primer set of pyc-F1 and pyc-R2, and the PCR product was sequence analyzed to confirm substitution of pyc gene. Hereinafter, the CGL (Δldh, ΔpoxB, Δpta-ackA, ΔactA, pyc^{P458S}) strain is referred to as a S006 strain.

4. Corynebacterium glutamicum (Δldh, ΔpoxB, Δpta-ackA, ΔactA, pyc^{P458S}, Ptuf::ppc)

C. glutamicum (Δldh, ΔpoxB, Δpta-ackA, ΔactA, pyc^{P458S}, Ptuf::ppc) was prepared as follows.

From the genomic DNA of C. glutamicum ATCC 13032, a PCR product was obtained by PCR amplification using a primer set of P246 (SEQ ID NO: 75) and P321 (SEQ ID NO: 76), and a PCR product was obtained by PCR amplification using a primer set of P324 (SEQ ID NO: 79) and P325 (SEQ ID NO: 80). A PCR product was obtained from a pGS-EX4 vector by PCR amplification using a primer set of P322 (SEQ ID NO: 77) and P323 (SEQ ID NO: 78). The three PCR products obtained therefrom were cloned at a location of a HindIII/EcoRI restriction enzyme of a pK19mobsacB vector to prepare a pK19mobsacB-Ptuf::ppc vector.

The pK19mobsacB-Ptuf::ppc vector was introduced to the S006 strain. PCR was performed by using a primer set of P250 (SEQ ID NO: 81) and P326 (SEQ ID NO: 82), and the PCR product was sequence analyzed to conform substitution of a promoter of a ppc gene to a promoter of a tuf gene. Hereinafter, the CGL (Δldh, ΔpoxB, Δpta-ackA, ΔactA, pyc^{P458S}, P_{tuf}::ppc) strain is referred to as a S054 strain.

5. Corynebacterium glutamicum (Δldh, ΔpoxB, Δpta-ackA, ΔactA, pyc^{P458S}, P_{tuf}::ppc, ΔpckG_P_{tuf}:: Ms.pck)

C. glutamicum (Δldh, ΔpoxB, Δpta-ackA, ΔactA, pyc^{P458S}, P_{tuf}::ppc, ΔpckG_P_{tuf}::Ms.pck) was prepared as follows.

From a genomic DNA of CGL ATCC 13032, a PCR product was obtained by PCR amplification using a primer set of P232 (SEQ ID NO: 83) and P242 (SEQ ID NO: 84), and a PCR product was obtained by PCR amplification using a primer set of P245 (SEQ ID NO: 87) and P288 (SEQ ID NO: 88). A PCR product was obtained from a pGS-EX4 vector by PCR amplification using a primer set of P243 (SEQ ID NO: 85) and P244 (SEQ ID NO: 86). The three PCR products obtained therefrom were in-fusion cloned at a location of a HindIII/EcoRI restriction enzyme of a pK19mobsacB vector to prepare a pK19mobsacB-Ptuf::pck vector.

A pckG gene (SEQ ID NO: 105) of M. succiniciproducens was obtained by synthesis, and this was cloned at a location of a BamHI/SalI restriction enzyme of a pUC57 vector (available from Thermoscientific) to obtain a pUC57-Ms.pck vector. From a genomic DNA of CGL ATCC 13032, a PCR product was obtained by PCR amplification using a primer set of P301 (SEQ ID NO: 92) and P302 (SEQ ID NO: 93), and, from the pK19mobsacB-Ptuf::pck vector, a PCR product was obtained by PCR amplification using a primer set of P232 (SEQ ID NO: 83) and P298 (SEQ ID NO: 89). Also, from the pUC57-Ms.pck vector, a PCR product was obtained by PCR amplification using a primer set of P299 (SEQ ID NO: 90) and P300 (SEQ ID NO: 91). The three PCR products obtained therefrom were in-fusion cloned at a location of a HindIII/EcoRI restriction enzyme of a pK19mobsacB vector to prepare a pK19mobsacB-ΔpckG_Ptuf::Ms.pck vector.

The pK19mobsacB-ΔpckG_Ptuf::Ms.pck vector was introduced to the S054 strain. PCR was performed by using a primer set of P303 (SEQ ID NO: 94) and P304 (SEQ ID NO: 95), and the PCR product was sequence analyzed to conform substitution of a promoter of a pckG gene to a promoter of a Ptuf::Ms.pck gene. Hereinafter, the CGL (Δldh, ΔpoxB, Δpta-ackA, ΔactA, pyc^{P458S}, P_{tuf}::ppc, ΔpckG_P_{tuf}::Ms.pck) strain is referred to as a S065 strain.

6. Corynebacterium glutamicum (Δldh, ΔpoxB, Δpta-ackA, ΔactA, pyc^{P458S}, P_{tuf}::ppc, ΔpckG_P_{tuf}::Ms.pck, ΔptsG)

C. glutamicum (Δldh, ΔpoxB, Δpta-ackA, ΔactA, pyc^{P458S}, P_{tuf}::ppc, ΔpckG_P_{tuf}::Ms.pck, ΔptsG) was prepared as follows.

From a genomic DNA of CGL ATCC 13032, a PCR product was obtained by PCR amplification using a primer set of ptsG_up_F and ptsG_up_R (SEQ ID NOS: 96 and 97), and a PCR product was obtained by PCR amplification using a primer set of ptsG_dn_F and ptsG_dn_R (SEQ ID NOS: 98 and 99). The two PCR products obtained therefrom were in-fusion cloned at a location of a HindIII/EcoRI restriction enzyme of a pK19mobsacB vector to prepare a pK19mobsacB-ΔptsG vector.

The pK19mobsacB-ΔptsG vector was introduced to the S065 strain. PCR was performed by using a primer set of ptsG-C_F and ptsG_C_R (SEQ ID NOS: 100 and 101), and the PCR product was sequence analyzed to conform deletion of the pckG gene. Hereinafter, the CGL (Δldh, ΔpoxB, Δpta-ackA, ΔactA, pycP458S, Ptuf::ppc, ΔpckG_Ptuf::Ms.pck, ΔptsG) strain is referred to as a S071 strain.

### EXAMPLE 1

### Confirmation of Effect of Succinic Acid on Production of Succinic Acid by Corynebacterium

An effect of succinic acid on production of succinic acid by Corynebacterium was confirmed by culturing Corynebacterium in the presence of succinic acid and measuring a glucose consumption rate and an amount of succinic acid production.

First, in order to confirm inhibition of glucose consumption and succinic acid production by succinic acid in Corynebacterium, a succinate inhibition assay was performed on a Corynebacterium S003 strain (Δldh, Δpta-ackA, ΔpoxB, and ΔactA).

In particular, for a seed culture, the strain was streaked on an active plate including agar 5 g/L of yeast extract, 10 g/L of beef extract, 10 g/L of polypeptone, 5 g/L of NaCl, and 20 g/L of agar, and then cultured at a temperature of 30° C. for about 48 hours. A single colony obtained therefrom was inoculated in 5 ml of a BHIS medium (including 37 g/L of brain-heart infusion agar, 91 g/L of D-sorbitol, pH 7.0) and grown overnight at a temperature of 30° C.

1 ml of the obtained culture solution was inoculated in a 25 ml of BHIS in a 250 ml flask and grown until an OD₆₀₀ value was 5.0. The culture solution was centrifuged, a supernatant thereof was removed to collect the microorganism only, and the microorganism was washed with CGXII minimal medium. The CGXII minimal medium includes 20 g/L of (NH₄)₂SO₄, 5 g/L of urea, 1 g/L of KH₂PO₄, 1 g/L of K₂HPO₄, 0.25 g/L of MgSO₄.7H₂O, 10 mg/L of CaCl₂, 10 mg/L of FeSO₄.7H₂O, 0.1 mg/L of MnSO₄.H₂O, 1 mg/L of ZnSO₄.7H₂O, 0.2 mg/L of CuSO₄.5H₂O, 20 mg/L of NiCl₂.6H₂O, 0.2 mg/L of biotin, 42 g/L of 3-morpholinopropanesulfonic acid (MOPS), and 4% (w/v) of glucose.

The resultant was inoculated in 20 ml of a minimal medium CGXII including succinic acid at a concentration of 0.00 M, 0.0625 M, 0.125 M, or 0.25 M until a cell concentration was OD₆₀₀=30, placed in an incubator (STX 40; Liconic instruments) in which an oxygen concentration is controlled, and then incubated while maintained the oxygen concentration at about 0% at 30° C.

The sample thus obtained was centrifuged and analyzed for concentration of succinic acid and glucose by HPLC.

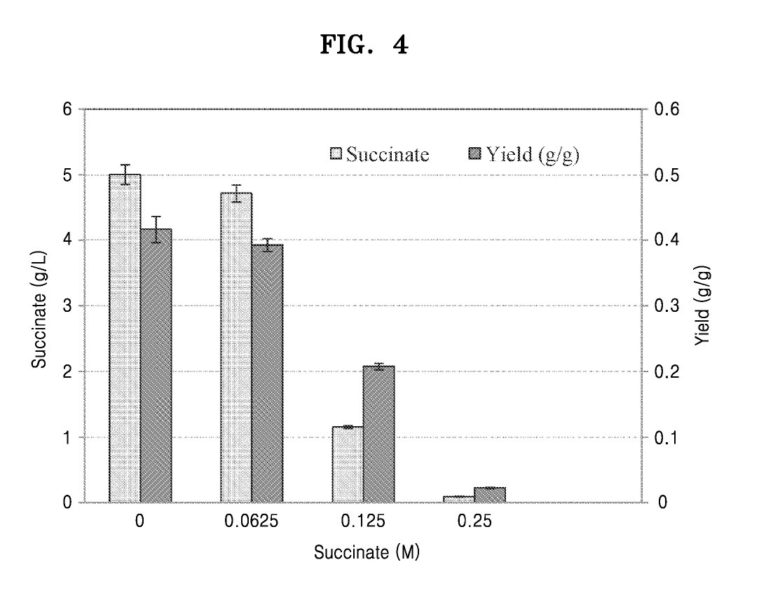

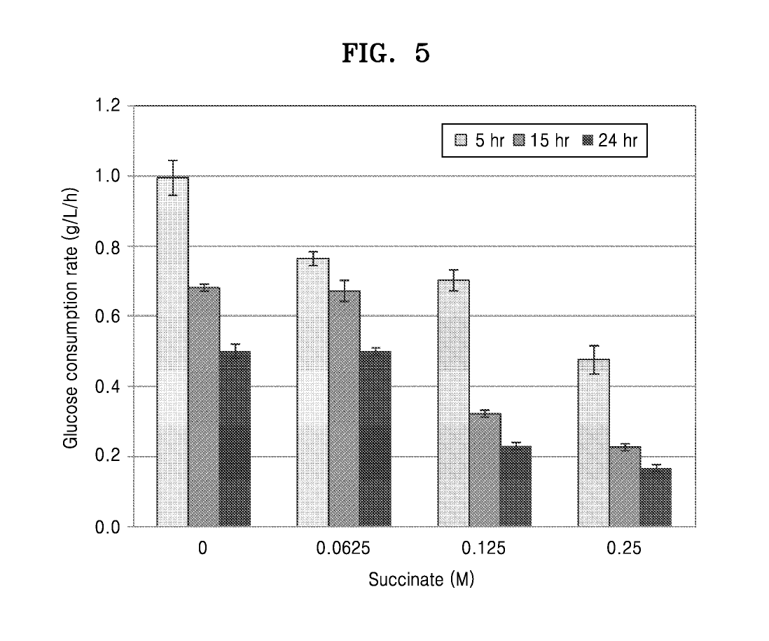

FIGS. 4 and 5 illustrate an amount of succinic acid production, a yield, and a glucose uptake rate of Corynebacterium glutamicum S003 strain according to concentration of succinic acid.

As shown in FIGS. 4 and 5, the glucose uptake rate and the amount of succinic acid production decreased according to an increase in concentration of succinic acid. When the concentration of succinic acid was 0.25 M, the glucose uptake rate decreased about 66.4%, and succinic acid was almost not produced. The IC₅₀ value of the glucose uptake rate was 0.11 M, that is, 12.98 g/L, and the IC₅₀ value of the succinic acid production was 0.1 M, that is, 11.8 g/L. From this result, it may be known that glucose uptake and production of succinic acid are suppressed by succinic acid in the succinic acid production using Corynebacterium.

### EXAMPLE 2

### Search for Gene Related to Suppression of Glucose Uptake and an Amount of Succinic Acid Production by Succinic Acid

In this example, the Corynebacterium glutamicum S003 strain was cultured in the presence of succinic acid, and the transcriptome thereof was analyzed by using a microarray to confirm change in the transcriptome caused by the presence of succinic acid.

First, in order to establish conditions for microarray analysis, a glucose uptake rate per time according to a concentration of succinic acid was confirmed. Samples were obtained from the strain cultured under the same flask test condition as in Example 1 after 5 hours, 15 hours, and 24 hours to confirm the glucose uptake rate.

As the result, glucose uptake was suppressed within the first 5 hours when 0.0625 M succinic acid was added to the medium, but a glucose uptake rate was the same with that of a strain in a control group after 15 hours (see FIG. 5). FIG. 5 illustrates a glucose uptake rate according to time when the Corynebacterium glutamicum ATCC S003 is cultured in the presence of succinic acid. This result may be understood that glucose uptake is suppressed by succinic acid at the beginning of the cell reaction.

Therefore, in order to minimize false-positive by a large amount of succinic acid, transcriptome analysis was performed by using cells that were cultured in an anaerobic condition for 5 hours in the medium, to which 0.0625 M succinic acid was added. After isolating total RNA from the cultured cells by using RNeasy midi kit (Qiagen), a transciptome was analyzed by using a Corynebacterium glutamicum ATCC 13032, 3×20K chip microarray (MYcroarray.com) according the protocol of the same manufacturer. 6134 unique probes are immobilized on the chip, where each of the probes is a three-times repeat.

As the result, it was confirmed that expression of genes increased or decreased 1.5 fold or more (p value≦Q.05) compared to that of the control group, to which succinic acid is not added. As the result of the transciptome analysis, most of genes involved in glycolysis process, TCA, and PPP pathways, except glpX, fda, pgk, mdh, and tpiA genes, did not have a significant difference in their expression. In the case of glpX, fda, pgk, mdh, and tpiA genes, their expressions were reduced about 1.91, 1.82, 2.29, 1.7, and 1.7 fold, each respectively. Also, it was confirmed that nine particular genes, that are NCgl1853, NCgl1855, NCgl0368, NCgl0275, NCgl2359, NCgl2404, NCgl2425, NCgl2988, and NCgl2877 and expected to be transcription regulators, had their expressions reduced 1.5 fold or more by succinic acid. This result is understood as succinic acid suppresses expression of the genes, and thus glucose uptake rate is reduced and production of succinic acid is suppressed accordingly. The fourteen genes are referred to as succinate-repressible genes (SRGs).

### EXAMPLE 3

### Confirmation of Effect of Overexpression of Fourteen Succinate-Repressible Genes on Glucose Uptake and Succinic Acid Production

The result of Example 1 implies that accumulation of succinic acid causes expression of the genes to be reduced, and thus succinic acid production and glucose uptake rate are suppressed. Thus, suppression of the gene expression by succinic acid is resolved by overexpressing the genes under the control of a tuf promoter, which is a constitutive promoter.

In this example, an effect of overexpression of the fourteen SRGs confirmed in Example 2 on glucose uptake rate and succinic acid production was confirmed.

A strain that overexpresses the fourteen SRGs was obtained by cloning the SRGs to Xhol/BamHI restriction site of a pGEX-4 vector to be expressed under an influence of a Ptuf promoter as described in “2. Preparation of pGEX_Ptuf::Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, and Ncgl1524 vector” under “Material and Method”, and then introducing each of the vectors thus obtained to a Corynebacterium glutamicum S003 strain.

The strains overexpressing the fourteen SRGs thus obtained were cultured under the same conditions with those in Example 1, and glucose uptake rates and amounts of succinic acid production were measured.

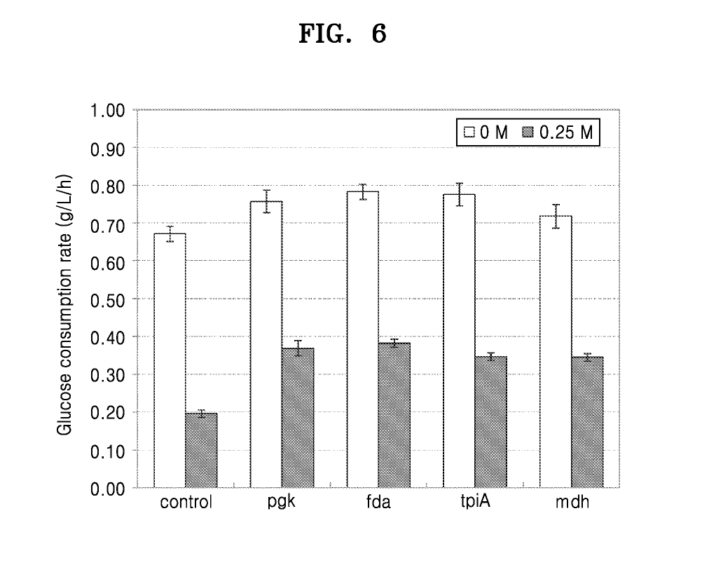

FIG. 6 illustrates a glucose uptake rate of a recombination strain in which glycolysis and TCA-related genes (pgk, mdh, fda, and tpiA) are overexpressed in the presence of 0 M and 0.25 M succinic acid.

As shown in FIG. 6, in the strain of overexpressing pgk, fda, tpiA, and mdh genes, the glucose uptake rates increased 1.76 fold or more compared to a glucose uptake rate of the control strain (S003, pGS-EX4) in the presence of 0.25 M succinic acid, but the glucose uptake rates were reduced 2.1 fold or more compared to a glucose uptake rate in the presence of 0 M succinic acid.

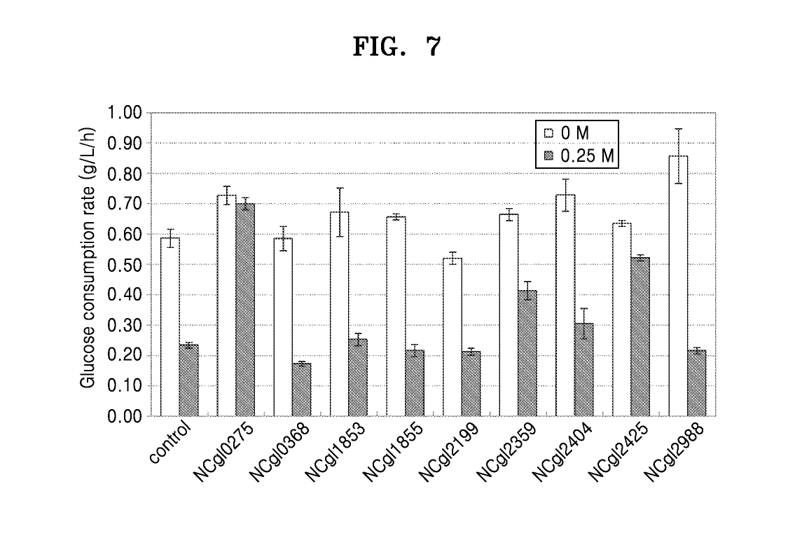

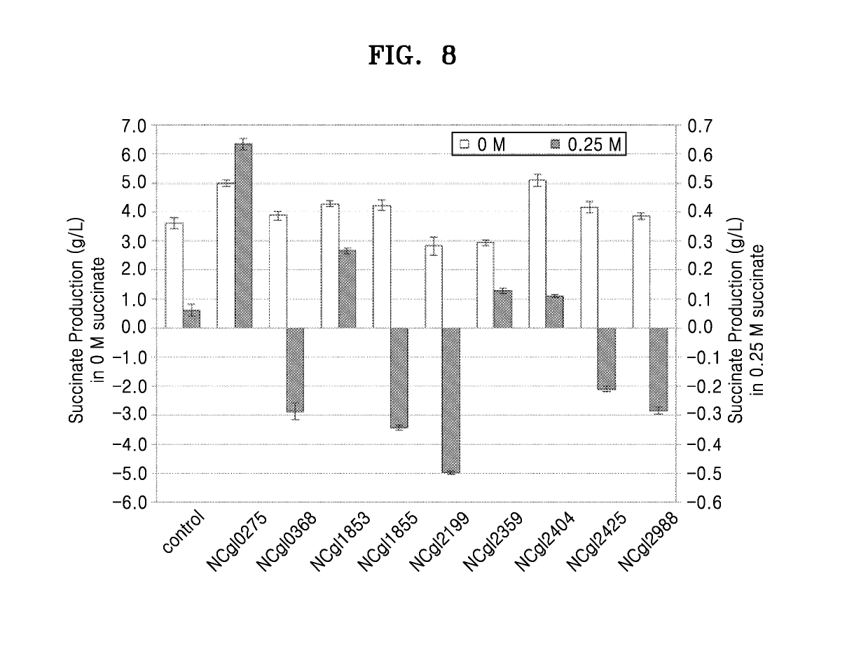

FIGS. 7 and 8 illustrate glucose uptake rates and amounts of succinic acid production when recombination strains, to which transcription regulator genes Ncgl1853, Ncgl1855, Ncgl2988, Ncgl2425, Ncgl2404, Ncgl0368, Ncgl2877, Ncgl0275, and Ncgl2359 were introduced, were cultured in the presence of 0 M and 0.25 M succinic acid.

As shown in FIG. 7, when the strains were cultured in the presence of 0 M succinic acid, NCgl1853, NCgl2359, NCgl2404, NCgl2425, NCgl0275, NCgl2988, and NCgl1855 overexpression strains had glucose uptake rates that were, each respectively, 15%, 13%, 24%, 8%, 24%, 81%, and 12% increased compared to glucose uptake rates of the control group, and NCgl1853, NCgl0368, NCgl2404, NCgl2425, NCgl0275, NCgl2988, and NCgl1855 overexpression strains had amounts of succinic acid production that were, each respectively, 18%, 7%, 41%, 15%, 27%, 7%, and 17% increased compared to an amount of succinic acid production of the control group.

As shown in FIG. 8, when the strains were cultured in the presence of 0.25 M succinic acid, NCgl1853, NCgl2359, NCgl2404, NCgl2425, and NCgl0275 overexpression strains had glucose uptake rates that were, each respectively, 8%, 76%, 30%, 122%, and 198%, and NCgl1853, NCgl2359, NCgl2404, and NCgl0275 overexpression strains had increased amounts of succinic acid production that were, each respectively, increased 4.37 fold, 2.10 fold, 1.79 fold, and 10.39 fold, compared to an amount of succinic acid production of the control group. Interestingly, in the case of a NCgl0275 overexpression strain, a glucose uptake rate was not significantly different in the presence of 0.25 M and 0 M succinic acid.

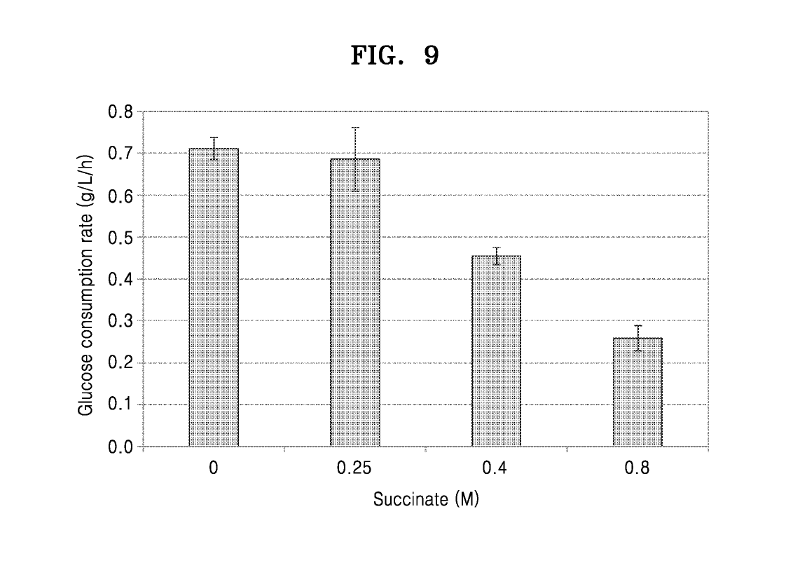

FIG. 9 illustrates a glucose uptake rate of a recombinant Corynebacterium strain in which a NCgl0275 gene is introduced to be overexpressed in the presence of succinic acid at a concentration of 0.25 M or higher. As shown in FIG. 9, the glucose uptake rate of the S003/pGEX4-NCgl0275 strain was tested by increasing a concentration of succinic acid, and as the result, the 10₅₀ value was 0.6 M (70.8 g/L) and this was about 5.45 fold increase compared to a glucose uptake of the control group. This result confirmed that suppression effect on succinic acid may be resolved by overexpression of the NCgl0275 gene, and that the NCgl0275 gene is an important regulatory factor with respect to the reduction of succinic acid production and glucose uptake rate.

### EXAMPLE 4

### Increase in Succinic Acid Production by Overexpression of NCgl0275 Gene

From Example 3, it was confirmed that the overexpression of NCgl0275 serves an important role resolving the suppression effect on succinic acid. Thus, a fed-batch fermentation was performed by using a S003/pGEX4-NCgl0275 in order to confirm an effect of the overexpression of NCgl0275 on succinic acid production. The fermentation was performed under the conditions described as follows.

The fermentation was performed by using an SF1 medium including 150 g/L of glucose (also including 3.5 g/L of corn-steep liquor, 2 g/L of (NH₄)₂SO₄, 1 g/L of KH₂PO₄, 0.5 g/L of MgSO₄.H₂O, 10 mg/L of FeSO₄.H₂O, 10 mg/L of MnSO₄.H₂O, 0.1 mg/L of ZnSO₄.7H₂O, 0.1 mg/L of CuSO₄.5H₂O, 3 mg/L of thiamin-HCl, 0.3 mg/L of D-Biotin, 1 mg/L of calcium pantothenate, and 5 mg/L of nicotinamide) in a 1.5-L fermentator. 5 mM of NH₄OH was used as a neutralizer to maintain pH at 7.3, and thus the fermentation was performed.

For seed culturing, S003/pGS-EX4 or S003/pGEX4-NCgl0275 strain was streaked on an active plate including 5 g/L of yeast extract, 10 g/L of beef extract, 10 g/L of polypeptone, 5 g/L of NaCl, and 20 g/L of agar, and cultured at 30° C. for 48 hours. A single colony obtained therefrom was inoculated in 5 ml of an S1 medium including 40 g/L of glucose, 10 g/L of polypeptone, 5 g/L of yeast extract, 2 g/L of (NH₄)₂SO₄, 4 g/L of KH₂PO₄, 8 g/L of K₂HPO₄, 0.5 g/L of MgSO₄.7H₂O, 1 mg/L of thiamin-HCl, 0.1 mg/L of D-biotin, 2 mg/L of Ca-pantothenate, and 2 mg/L of nicotineamide, and cultured at 30° C. until an OD₆₀₀ value was 5.0. The culture solution was transferred to 35 ml of a S1 medium and cultured at 30° C. for 5 hours.

The seed culture solution was inoculated in 350 ml of an SF1 medium and cultured at a rate of 500 rpm, 0.2 vvm (=aeration volume/medium volume/minute), until an OD₆₀₀ value was 70. Then, magnesium carbonate (Sigma M5671) was added thereto a final concentration 0.2 M, and aeration was lowered to 0 vvm so that the fermentation was performed under an anaerobic condition. Sampling was performed every hour, and the samples were diluted at a 1/100 concentration to analyze an organic acid by HPLC.

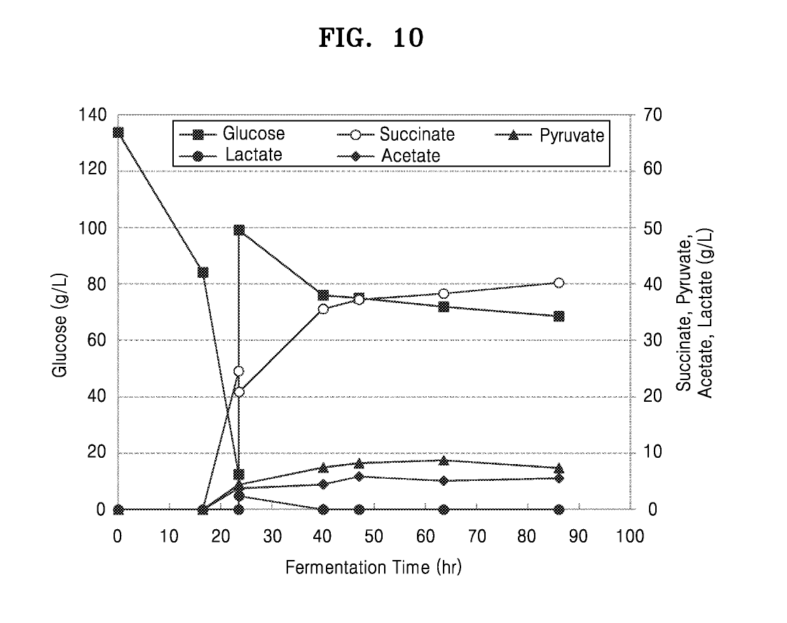

FIG. 10 shows the fermentation result by using a recombined Corynebacterium glutamicum S003 strain. As shown in FIG. 10, the S003/pGS-EX4 strain produced 40.2±1.2 g/L of succinic acid, 7.4 g/L of pyruvate, and 5.5 g/L of acetate. A succinic acid production yield was 0.37 g/g, and a succinic acid productivity was 0.58 g/L/h.

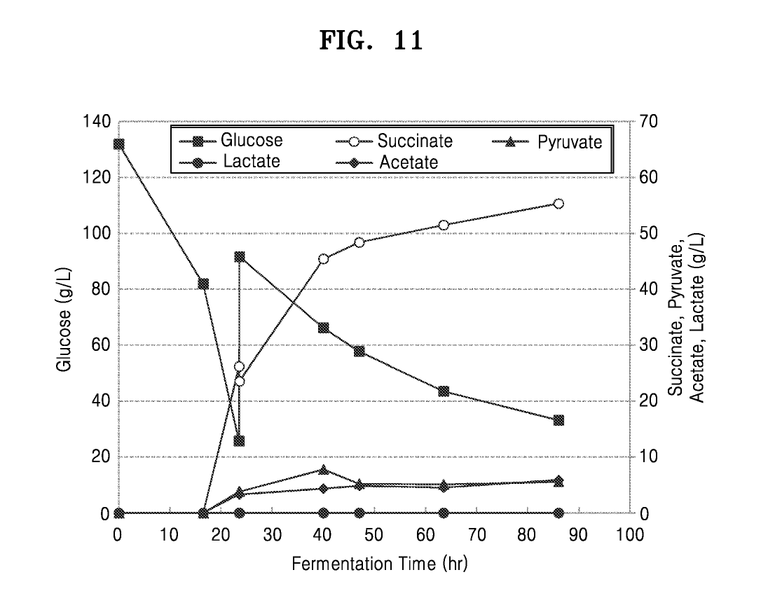

FIG. 11 shows the fermentation result by using a NCgl0275 gene-overexpressed recombined Corynebacterium glutamicum S003 strain. As shown in FIG. 11, the S003/pGEX4-NCgl0275 strain produced 55.36±1.8 g/L of succinic acid as a final concentration, and this was 37.7% increased amount of succinic acid production compared to that of the control group. Also, succinic acid production yield was increased to 0.53 g/g, and a succinic acid productivity was increased to 0.8 g/L/h. In particular, in the case of the S003/pGS-EX4 strain after 40 hours of fermentation, its glucose uptake rate and succinic acid productivity dropped, but in the case of the NCgl0275 overexpressed strain, its glucose uptake rate was 0.72 g/L/h and a succinic acid productivity was 0.22 g/L/h after 40 hours of fermentation. This may be resulted because suppression effect on succinic acid production is resolved by overexpression of the NCgl0275 gene.

### EXAMPLE 5

### Metabolic Engineering for Increase in Production of Succinic Acid

S006, S065, and S071 strains were prepared by using a metabolic engineering method to increase succinic acid production of the strains, and abilities of producing succinic acid in the strains were confirmed through a flask test.

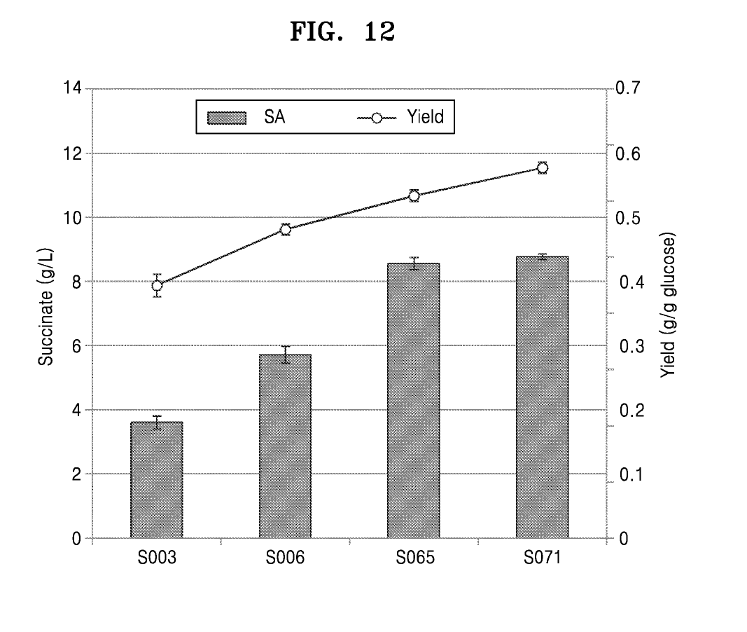

FIG. 12 shows amounts of succinic acid production and yields of the recombined Corynebacterium S003, S006, S065, and S071 strains. As shown in FIG. 12, the S006 strain consumed 12.6±0.32 g/L of glucose and produced 5.71±0.26 g/L of succinic acid (yield=0.45±0.01 g/g). The S065 strain, in which an anaplerotic pathway is enhanced, had 30% increased glucose uptake (16.64±0.04 g/L) and 50% increased amount of succinic acid (8.56±0.19 g/L, yield=0.51±0.02 g/g), compared to those of the S006 strain. Also, the S065 strain had 80% reduced pyruvate accumulation compared to that of the S006 strain. Lastly, the S071 strain prepared by deleting a ptsG gene had the same level of succinic acid production but had 9.8% increased succinic acid production yield (0.56±0.01 g/g), compared to those of the S065 strain.

### EXAMPLE 6

### Fermentation of S071/pGEX4-NCgl0275 Strain

The S071 strain was fermented under the same conditions as in Example 4 to confirm an effect of NCgl0275 overexpression by introducing pGEX4-NCgl0275 to the S071 strain.

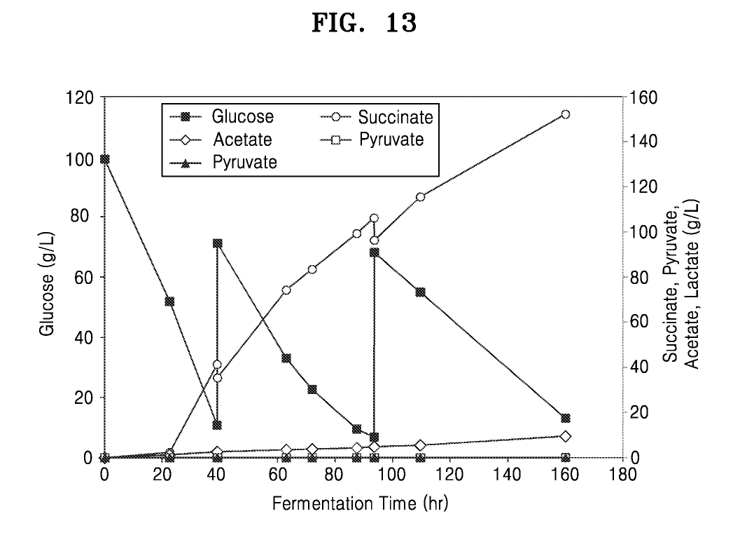

FIG. 13 shows the fermentation result of the NCgl0275 gene-overexpressed recombinant Corynebacterium S071 strain. As shown in FIG. 13, the S071/pGEX4-NCgl0275 strain after the fermentation consumed 139 g/L of glucose and produced 152.2 g/L as a final succinic acid productivity. Interestingly, a yield of the strain within an anaerobic period was 1.1 g/g (1.67 mol/mol), which was almost at the maximum theoretical yield (1.71 mol/mol). This has been the highest titer and yield in record among conventionally known succinic acid production strains.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present disclosure have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A genetically engineered bacterial cell comprising increased activity of an expression product of a gene having about 95% or more sequence identity to at least one of Ncgl1853, Ncgl1855, Ncgl2988, Ncgl2425, Ncgl2404, Ncgl0368, Ncgl2877, Ncgl0275, Ncgl2359, Ncgl1525, Ncgl0976, Ncgl2673, Ncgl2297, or Ncgl1524, wherein the activity is increased in comparison to a non-engineered bacterial cell of the same type; and
increased glucose consumption rate, increased succinic acid productivity, or both as compared to a non-engineered bacterial cell of the same type.

2. The genetically engineered bacterial cell of claim 1, wherein the cell is cultured in the presence of succinic acid.

3. The genetically engineered bacterial cell of claim 1, wherein the increase in activity of the expression product of the gene is due to increased expression of the gene, derepression of the gene expression depression caused by succinic acid, increased specific activity of the expression product, or combination thereof.

4. The genetically engineered bacterial cell of claim 1, wherein the genetically engineered bacterial cell produces succinic acid under micro-aerobic or anaerobic conditions.

5. The genetically engineered bacterial cell of claim 1, wherein the cell is of the Corynebacterium genus.

6. The genetically engineered bacterial cell of claim 1, wherein the increased activity of the expression product is caused by an increased copy number of the gene encoding the expression product or by modification of the regulatory sequence of the gene encoding the expression product.

7. The genetically engineered bacterial cell of claim 6, wherein the genetically engineered bacterial cell comprises an exogeneous polynucleotide that encodes the expression product, or amplification of an internal gene that encodes the expression product.

8. The genetically engineered bacterial cell of claim 1, wherein the genetically engineered bacterial cell comprises a mutation in the gene encoding the expression product that causes the increase in activity of the expression product.

9. The genetically engineered bacterial cell of claim 1, wherein the expression product comprises an amino acid sequence having about 95% or more sequence identity to at least one of SEQ ID NOS: 1 to 14.

10. The genetically engineered bacterial cell of claim 1, wherein the gene encoding the expression product has about 95% or more sequence identity to at least one of SEQ ID NOS: 15 to 28.

11. The genetically engineered bacterial cell of claim 1, wherein an L-lactate dehydrogenase gene, a pyruvate oxidase gene, a phosphotransacetylase gene, an acetate kinase gene, an acetate CoA transferase gene, or a combination thereof is deleted or disrupted in the genetically engineered bacterial cell.

12. The genetically engineered bacterial cell of claim 1, wherein activity of a pyruvate carboxylase catalyzing conversion of pyruvate to oxaloacetate is increased in the genetically engineered bacterial cell as compared to a non-genetically engineered bacterial cell of the same type.

13. The genetically engineered bacterial cell of claim 12, wherein the pyruvate carboxylase has P458S substitution at SEQ ID NO: 14.

14. The genetically engineered bacterial cell of claim 12, wherein activity of pckG catalyzing conversion of PEP to OAA is increased in the genetically engineered bacterial cell as compared to a non-genetically engineered bacterial cell of the same type.

15. A method of producing succinic acid, the method comprising:
culturing the bacterial cell of claim 1; and
collecting succinic acid from the culture.

16. The method of claim 15, wherein the culturing is performed under a microaerobic or an anaerobic condition.

17. The method of claim 15, wherein the culturing is performed at a pH of about 6 to about 8.

18. The method of claim 15, wherein the bacterial cell is of the Corynebacterium genus.

19. The method of claim 15, wherein an L-lactate dehydrogenase gene, a pyruvate oxidase gene, a phosphotransacetylase gene, an acetate kinase gene, an acetate CoA transferase gene, or a combination thereof is deleted or disrupted in the bacterial cell.

20. The method of claim 15, wherein activity of a pyruvate carboxylase catalyzing conversion of pyruvate to oxaloacetate is increased in the bacterial cell as compared to a non-genetically engineered bacterial cell of the same type.
